(19) United States
(12) Patent Application Publication
(10) Pub. No.: US 2003/0186300 A1
(43) Pub. Date: Oct. 2, 2003

(54) Methods and products for modulation of reproductive processes and for diagnosis, prognostication and treatment of related conditions

(76) Inventor: Ali Akoum, Ste-Foy (CA)

Correspondence Address: SMART & BIGGAR, Suite 3400, 1000 dela Gauehetlere Street West, Montrea, QC, H3B 4W5, CA

(21) Appl. No.: 10/391,808

(22) Filed: Mar. 20, 2003

Related U.S. Application Data
(60) Provisional application No. 60/367,764, filed on Mar. 28, 2002.

(30) Foreign Application Priority Data
Mar. 20, 2002 (CA) 2,377,786

(51) Int. Cl. C12Q 1/68; G01N 33/53; G01N 33/567

(52) U.S. Cl. 435/6; 435/7.2

(57) ABSTRACT

The invention relates to methods, commercial packages and compositions for the diagnosis, prognostication and treatment of reproduction-associated diseases. The invention also relates to to methods, commercial packages and compositions for the determination and modulation of endometrial receptivity.

This application claims the benefit of U.S. provisional application No. 60/367,764 filed Mar. 28, 2002, incorporated herein by reference in its entirety.

### FIELD OF THE INVENTION

The invention relates to diagnostic, prognostic, therapeutic and/or predictive methods and products which may be used in respect of reproductive processes such as fertility, and associated conditions,-such as endometriosis and infertility.

### BACKGROUND OF THE INVENTION

Both MCP-1 and IL-1RII have been studied in respect of the endometrium (see appendices 1 and 2 below).

IL-1 and IL-1RII in the endometrium

Interleukin-1 (IL-1) is the term used to describe two polypeptides (IL-1a and IL-1β) that play a key role in immune and inflammatory reactions (Dinarello C A, 1996). Three receptors for IL-1, type I (IL-1R1), type II (IL-1RII), and type III (IL-1RIII), have been identified in different cell types (Dinarello C A, 1996; Arend W P, 1991). Cell activation in response to IL-1 appears to be mediated exclusively via the IL-1R1 (Sims J E, et al, 1988; Sims J E, et al, 1993), with coexpression of a receptor accessory protein (IL-1R-AcP or IL-1RIII) being crucial to IL-1-mediated signaling events (Greenfeder S A, et al, 1995; Wesche H, et al, 1997; Korherr C, et al, 1997). In itself, IL-1RII is not a signaling molecule but, in fact, is reported to be a decoy target of IL-1 (McMahan C J, et al, 1991; Colotta F, et al, 1994). Additionally, IL-RII could be shed from the cell surface as a soluble molecule that would then capture IL-1 and inhibit its binding to IL-1RI (Giri J G, et al, 1990; Symons J A, et al, 1995; Bossu P, et al, 1995).

It has been shown that IL-1 is secreted by human blastocysts, and it is thought to act as an embryonic signal (Baranao R I, 1992). The cytokine was also detected locally in the endometrial tissue during the late secretory phase of the menstrual cycle (Kauma S, et al, 1990). Expression of the functional receptor of IL-1 (i.e., IL-1RI) has been detected in the human endometrium as well (Simon C, et al, 1993; Bigonnesse F, et al, 2001), where it appears to play a key role in the implantation process (Simon C, et al, 1994).

Due to its pleiotropic activity and potent proinflammatory effects, IL-1 is tightly regulated in the body by complex control systems. In particular, two inhibitors participate in these regulatory mechanisms: the receptor antagonist (IL-1ra), which binds avidly to IL-1RI and prevents IL-1 binding and signal transduction; and IL-1RII, which is considered to be a natural scavenger for IL-1. IL-1RII can very efficiently bind IL-1β, whereas its affinity for IL-1α and IL-1ra is 10- to 100-fold lower (Boraschi D, et al, 1995).

IL-1 has been shown to be involved in numerous immunological and reproductive activities occurring normally in the human endometrium during a normal menstrual cycle or during embryonic implantation and development (Simon C, et al, 1995). A growing body of evidence indicates that IL-1 may play an important role in the pathophysiology of, endometriosis, a gynecological disease that is believed to arise from ectopic growth of endometrial tissue and is associated with a chronic immunoinflammatory process (Halme J, et al, 1984; Grosskinsky C M, et al, 1993; Sahakian V, et al, 1993). In women with endometriosis, peripheral blood monocytes (Zeller J M, et al, 1989) as well as peritoneal macrophages (Mori H, et al, 1992) were found to be more activated than in normal women and to secrete elevated levels of IL-1. Increased concentrations of IL-1 were detected in the peritoneal fluid of women suffering from endometriosis (Mori H, et al, 1992; Fakih H, et al, 1987). According to our previous data, IL-1 enhances the production of monocyte chemotactic protein-i (MCP-1) by human endometriotic cells (Akoum A, et al, 1995) and by eutopic endometrial cells of women with endometriosis (Jolicoeur C, et al, 1998). Moreover, these cells appeared to be more sensitive to the action of IL-1 in women with than in women without endometriosis (Akoum A, et al, 1995).

Three receptors for IL-1, now designated as IL-1RI, IL-1RII, and IL-1RIII (more commonly called IL-1R AcP [accessory protein]), have been described. The relative importance of these receptors in IL-1 signaling has been recently clarified. A critical role for the IL-1RI and IL-1R AcP in IL-1-induced cell activation has been demonstrated by several groups (Colotta F, et al, 1993; Sims J E, et al, 1993; Greenfeder S A, et al, 1995). In contrast, IL-1RII appears to be dispensable for IL-1 signaling and may act as a decoy receptor (Colotta F, et al, 1993; Sims J E, et al, 1993). Interleukin-1 receptor antagonist (IL-1ra) is another natural inhibitor of IL-1, which competes with IL-1α and IL-1β for IL-1 RI (Granowitz E V et al, 1991). Results of several studies indicate that the IL-1 system is available locally in the human endometrial tissue and may be an important mediator in local cellular interactions (Tabibzadeh S, et al, 1990; Simon C, et al, 1993; Bigonnesse F, et al, 2001; Kauma S, et al, 1990; Bigonnesse F, et al, 2001). According to Sahakian et al. (Sahakian V, et al, 1993), ectopic endometrial tissue does not express IL-1ra.

Endometriosis is an immune-related chronic inflammatory disease, characterized by the presence of endometrial-like tissue in ectopic locations, mainly in the peritoneal cavity, and associated with increased secretion of proinflammatory cytokines including IL-1, IL-6, IL-8, tumor necrosis factor-alpha (TNF-α) and MCP-1 in the peritoneal fluid (Senturk L M, et al, 1999; Mulayim N, et al, 1999). These factors have been postulated as being implicated in the development and progression of the disease. Immunoinflammatory changes observed in patients with endometriosis are not restricted only to the peritoneal cavity where endometriotic lesions frequently develop (Senturk L M, et al, 1999; Mulayim N, et al, 1999; Harada T, et al, 2001), but were also detected in the eutopic endometrium (Braun D P, et al, 1998; Sharpe-Timms K L, et al, 2001)), and the peripheral blood (Mathur S P, 2000; Dmowski W P, et al, 1994). In endometriosis, peritoneal macrophages are more activated and secrete elevated concentrations of proinflammatory cytokines (Mori H, et al, 1991; Rana N, et al, 1996). However, other reports indicate that peripheral blood monocytes from women with endometriosis are also activated (Zeller J M, et al, 1989;Braun D P, et al, 1996) and show the ability to stimulate endometrial cell growth in vitro, whereas monocytes of normal fertile women suppress the proliferation of these cells (Braun D P, et al, 1994). However, the mechanisms involved in the activation of these cells remain unknown.

It has previously been shown that IL-RII presents defective expression in the endometrium of women with endometriosis (Akoum A, et al, 2001).

The etiology of endometriosis is still not clearly defined. Genetic predisposition, environmental toxins, hormonal factors, and immune deficiency may contribute to the susceptibility of a woman to develop this disease (McLaren J, 2000). However, a key condition for endometrial tissue to survive and grow ectopically following successful adhesion and implantation is the establishment of an effective new blood supply, a process involving the generation of new blood vessels or angiogenesis (Healy D I, et al, 1998).

Early and most active endometriotic lesions are markedly vascularized; increased vascularization is seen at the implant surface and also in the surrounding peritoneal, tissue (Wiegerinck M A, et al, 1993) suggesting that endometriotic implant is capable of inducing its own neovascularization by deriving local microvasculature.

There is therefore a need to better characterize these phenomena to identify new methods and materials useful for the the modulation of reproductive processes and the treatment, diagnosis and prognostication of associated disease.

### SUMMARY OF THE INVENTION

In a first aspect, the invention provides a method of assessing a reproduction-associated disease in a subject, said method comprising: determining a test level of a parameter selected from the group consisting of MIF protein; MIF encoding RNA, and MIF activity; in a tissue or body fluid from said subject; and comparing said test level to an established standard; or to a corresponding level of said parameter in a tissue or body fluid of a control subject; or to a corresponding level of said parameter in a tissue or body fluid obtained from said subject at an earlier time; wherein an increase in said test level is indicative of reproduction-associated disease.

The invention further provides a method of assessing endometrial receptivity in a subject, said method comprising determining, in said subject, a test level of a parameter selected from the group consisting of MIF protein; IL-1RII protein; MIF encoding RNA; IL-1RII encoding RNA; MIF activity; and and IL-1RII activity; wherein said test level is indicative of endometrial receptivity. In an embodiment, the method further comprises comparing said test level with an established standard, or to a corresponding level of said parameter from a control subject; or to a corresponding level of said parameter obtained from said subject at an earlier time, to obtain a comparison result. In a further embodiment, the method further comprises predicting a window of implantation in accordance with said comparison result. In an embodiment, the subject is in a secretory phase of a menstrual cycle. In an embodiment, said test level is determined in a body fluid or a tissue obtained from said subject.

The invention further provides a method of assessing a reproduction-associated disease in a subject, said method comprising: (a) determining a test level of a parameter selected from the group consisting of IL-1RII protein; IL-1RII encoding RNA; and IL-1RII activity; in a sample (e.g. serum) from said subject; and (b) comparing said test level to an established standard; or to a corresponding level of said parameter in a sample (e.g. serum) of a control subject; or to a corresponding level of said parameter in a sample (e.g. serum) obtained from said subject at an earlier time; wherein a decrease in said test level is indicative of reproduction-associated disease.

The invention further provides a method of preventing or treating a reproductive-related disease in a subject, said method comprising: (a) administering an effective amount of an IL-1RII or an IL-1RII-related compound to said subject; (b). enhancing IL-1RIT activity in said subject; (c). enhancing IL-1RII expression in a cell or tissue of said subject; and any combination of (a) to (c). In an embodiment, the cell is a endometrial cell, and/or the tissue is an endometrial tissue.

The invention further provides a commercial package comprising means for assessing the level of a parameter selected from the group consisting of: MIF protein; MIF encoding RNA; and MIF activity; in a tissue or body fluid; together with instructions for diagnosis, prognostication, or both, of reproduction-associated disease.

The invention further provides a commercial package comprising means for assessing the level of a parameter selected from the group consisting of: MIF protein; IL-1RII protein; MIF encoding RNA; IL-1RII encoding RNA; MIF activity; and IL-1RII activity; in a tissue or body fluid, together with instructions for the determination of the endometrial receptivity.

The invention further provides a commercial package comprising means for assessing the level of a parameter selected from the group of: IL-1RII protein; IL-1RII encoding RNA; and IL-1RII activity; in serum, together with instructions for the diagnosis, or prognostication, or both, of reproduction-associated disease.

In an embodiment, the above noted commercial packages further comprise a reference value or reference sample of said parameter. In an embodiment, the reference value is an established standard of said parameter; and wherein the reference sample is a corresponding level of said parameter in a tissue or body fluid of a control subject.

The invention further provides a composition for preventing or treating a reproduction-associated disease in a subject, said composition comprising an IL-1RII or an IL-1RII related compound in admixture with a pharmaceutically acceptable carrier.

The invention further provides a composition for lowering endometrial receptivity in a subject, said composition comprising an IL-1RII or an IL-1RII related compound in admixture with a pharmaceutically acceptable carrier.

The invention further provides a commercial package comprising an IL-1RII or an IL-1RII related compound together with instructions for treating a reproductive-related disease in a subject.

The invention further provides a method for lowering endometrial receptivity in a subject, said method comprising administering an effective amount of an IL-1RII or IL-1RII related compound to said subject.

The invention further provides a commercial package comprising an IL-1RII or an IL-1RII related compound together with instructions for lowering endometrial receptivity in a subject.

In an embodiment, the subject is a mammal, in a further embodiment, a human. In an embodiment, the body fluid or tissue is endometrial tissue or a derivative thereof. In an embodiment, the reproduction-associated disease is endometriosis or infertility.

### BRIEF DESCRIPTION OF THE DRAWINGS

FIG. 1. Distribution of MCP-1 concentrations in peritoneal fluid as described in (Akoum A et al, 1996). Distribution of MCP-1 concentrations in peritoneal fluid of normal controls (n=44) and patients with endometriosis (n=57) according to stage of disease. Horizontal bars, Medians of MCP-1 concentrations; horizontal dashed line, cutoff value (100 μg/ml).

FIG. 2. Chemotactic response of U937 cells to plasma from normal controls and patients with endometriosis as described in (Akoum A. et al, 1996). U937 cells were stimulated with dibutryl cyclic adenosine monophosphate to induce differentiation and used at 600×10³ cells per well. Results were expressed as mean number of cells that migrate to lower side of membrane per well ±SEM. Biologic activity of MCP-1 was evaluated by preincubating plasma samples with polyclonal rabbit anti-MCP-1 antibody (1:500 dilution) for 30 minutes at 37° C. before addition of differentiated U937 cells to top wells. Preimmune rabbit serum was assayed in same fashion without repression of activity. N-Formyl-methionyl-leucyl-phenylalanine (FMLP). 10⁻⁷ mol/L and phosphate-buffered saline solution were used as positive and negative controls, respectively. N, Normal; E, endometriosis. Asterisk, p<0.05, versus control; two asterisks, p<0.01, versus control.

FIG. 3. Representative illustration of MCP-1 immunostaining in the endometrium as described in (Jolicoeur C et al, 1998). Representative illustration of MCP-1 immunostaining in the endometrium of normal controls (A, proliferative day 9, immunostaining score 1; B, secretory day 19, immunostaining score 1) and patients with endometriosis (C, proliferative day 8, immunostaining score 2; D, secretory day 24, immunostaining score 2). Note the brown positive immunostaining in endometrial glands of women with endometriosis, particularly in the secretory phase, compared with that of normal subjects. Magnification ×333.

FIG. 4. Detection of MCP-1 mRNA in the endometrium by in situ hybridization as described in (Jolicoeur et al, 1998). Sections were hybridized with biotin-labeled cDNA probes. Biotin detection was performed using a rabbit polyclonal anti-biotin antibody, a biotinylated-goat anti-rabbit polyclonal antibody, and fluorescein isothiocyanate-conjugated streptavidin, respectively. Slides were treated with propidium iodine, which makes the nucleus visible in yellow-orange upon UV excitation, and mounted in the presence of an anti-fading agent (p-phenylenediamine). Appearance of endometrial glands (g) and stroma (s) at ×167 (A1) and ×666 (A2) magnification following hybridization, and staining with propidium iodine. Note the green-yellow hybridization signal (arrow) that could only be observed at ×1665 magnification (B) predominantly in endometrial glands.

FIG. 5. Representative illustration of MCP-1 mRNA expression in the endometrium as decribed in (Jolicoeur et al, 1998). Representative illustration of MCP-1 mRNA expression in the endometrium of normal controls (A, proliferative day 13, score 1; B, secretory day 22, score 1) and patients with endometriosis (C, proliferative day 12, score 2; D, secretory day 25, score 2). Note the positive green-yellow spots (arrows) in endometrial glands of women with endometriosis, particularly in the secretory phase, compared with that of normal subjects. Magnification, ×1665.

FIG. 6. Representative illustration of IL-1RII immunostaining in the human endometrium as described in (Akoum A et al, 2001). Sections of endometrial tissue were incubated with mouse monoclonal anti-IL-1RII antibody (A, proliferative day 13; B, secretory day 24; original magnification, ×68) or with an equivalent concentration of normal mouse TgGs (C and D, respectively; original magnification, ×68). Sections were then incubated successively with biotinylated goat anti-mouse polyclonal antibody and avidin-biotinylated horseradish peroxidase complex. The immunoreaction was revealed with diaminobenzidine (brown staining) and hematoxylin was used for counterstaining (blue staining). Note the brown fine positive staining in stromal and epithelial cells (cellular staining) (E-H; original magnification, ×268), and the brown deposit (arrow) that is primarily located at the apical side of glandular (E, secretory phase day 24) and surface (F, secretory phase day 16) epithelium, or more spread within the glands lumen (G, secretory phase day 16). Positive immunostaining is also detected in isolated stromal cells (c) (G, secretory phase day 16) and microvessels (v) (H, secretory phase day 24) found in the stroma in the secretory phase of the menstrual cycle. s=stroma, g=gland.

FIG. 7. Dual immunofluorescent staining of IL-1RII and IL-1β in the endometrial tissue of normal women as described in (Akoum A et al, 2001). Dual immunofluorescent staining of IL-1RII (A) and IL-1β (B) in the endometrial tissue of normal women. Tissue sections were successively incubated with mouse monoclonal anti-IL-1RII antibody, rabbit polyclonal anti-IL-1 antibody, and biotinylated goat anti-rabbit antibody before being incubated simultaneously with rhodamine-conjugated goat anti-mouse antibody and fluorescein isothiocyanate-conjugated streptavidin. Serial sections incubated with normal mouse and normal rabbit IgGs instead of the primary antibodies were included as negative controls (C and D) (original magnification, ×160). Note the co-expression of IL-1RII (red color) and IL-1β (green-yellow color) within the luminal deposit in endometrial glands. Data from a normal woman in the secretory phase of the menstrual cycle (day 23).

FIG. 8. Representative illustration of IL-1RII immunostaining in the endometrium of women with and without endometriosis as described in (Akoum A et al, 2001). A) Normal secretory day 24, luminal staining score 3, cellular staining score 2 in stromal and epithelial cells. B) Endometriosis stage secretory day 26, luminal staining score 0, cellular staining score 2 in stromal and epithelial cells. Note the lack of endometrial glands in B. Original magnification ×160.

FIG. 9. Western blot analysis of IL-1RII expression in the endometrial tissue as described in (Akoum A et al, 2001). A) Normal women: day 10 (lanes 1 and 3) and day 24 (lanes 2 and 4). B) Women with endometriosis: stage II, day 13 (lanes 1 and 3) and stage II, day 25 (lanes 2 and 4). Equal amounts of endometrial proteins (100 μg/lane) were subjected to sodium dodecyl sulfate-polyacrylamide gel electrophoresis and transferred to nitrocellulose membranes. IL-1RII was detected using a mouse monoclonal antibody (lanes 1 and 2) and the immunocomplex was revealed by chemiluminescence. No immunoreaction was observed in negative controls where the anti-IL-1RII antibody was replaced by an equal concentration of mouse immunoglobulins of the same isotype (lanes 3 and 4).

FIG. 10. Immunohistochemical detection of IL-1RII in the endometrium. Immunohistochemical analysis of the expression IL-1RII in the endometrium was performed. A) Positive brown immunostaining in the glands and the stroma (Day 24). B) Negative control: serial section from the same endometrial tissue incubated with normal mouse immunoglobulins instead of the primary antibody. C-F) Representative illustrations of IL-1RII intensity of staining in the endometrium throughout the menstrual cycle: early proliferative phase, Day 6 (C); late proliferative phase, Day 13 (D); midsecretory phase, Day 18 (E); and late secretory phase, Day 23 (F). Note the marked immunostaining in early proliferative (C) and late secretory (F) endometrial tissues and a reduced intensity of that staining in the glands and surface epithelium of midsecretory phase endometrial tissue (E). Magnification ×290.

FIG. 11. Graphical illustration of immunostaining scores of IL-1RII and their distribution in the endometrium according to the day of the menstrual cycle. Immunostaining scores of IL-1RII were comprared according to the day of the menstrual cycle. A) Luminal staining in glandular and surface epithelia. B) Cellular staining in glandular and surface epithelia. C) Cellular staining in the stroma. Stromal extracellular staining could not be evaluated and was detectable only in late secretory endometrial tissues. Vertical hatched lines represent threshold days separating the cycle into different expression periods according to the intensity of IL-1RII immunostaining.

FIG. 12. Analysis of IL-1RII protein expression in the endometrium according to the day of the menstrual cycle. Western blot analysis was performed on endometriotic tissues during different day of the menstrual cycle. A) Western blot analysis of IL-1RII protein expression in the endometrium throughout the menstrual cycle: Day 6 (lanes 1 and 5), Day 13 (lanes 2 and 6), Day 19 (lanes 3 and 7), and Day 26 (lanes 4 and 8). The antibody specifically recognized different bands, the molecular weights of which range from 68 to 31 kDa. The immunoreactive bands (lanes 1-4) were absent when the primary mouse monoclonal anti-IL-1RII antibody was replaced by an equal concentration of normal mouse IgGs (lanes 5-8). Although barely detectable in the early proliferative phase (lane 1), IL-1RII bands were markedly intense at the approach of ovulation (lane 2), but their intensity clearly decreased in the midsecretory phase (lane 3) and increased again thereafter in late secretory endometrial tissue (lane 4). B) Nitrocellulose membrane-transferred proteins were incubated with the antibody in the absence (lanes 1 and 2) or the presence (lanes 3 and 4) of an excess of rIL-1RII (20 mg/ml). Minor bands recognized by the antibody disappeared following competitive inhibition by recombinant IL-1RII, whereas the intensity of major higher molecular weight bands was considerably reduced. Endometrial tissues were at Day 14 (lanes 1 and 3) and Day 28 (lanes 2 and 4) of the menstrual cycle.

FIG. 13. Localization of IL-1RII mRNA in the endometrium by in situ hybridization. Sections were hybridized with biotin-labeled cDNA probes, detection of biotin was performed using a rabbit polyclonal antibiotin antibody, a biotinylated goat anti-rabbit polyclonal antibody, and fluorescein isothiocyanate-conjugated streptavidin, respectively and slides were treated with propidium iodine, which makes the nucleus visible in yellow-orange on ultraviolet excitation, and mounted in the presence of an antifading agent (p-phenylenediamine). Appearance of endometrial glands (A), surface epithelium (B), and stroma (C) are shown at ×167 (1) and ×666 (2) magnification following hybridization and staining with propidium iodine. Note the green-yellow hybridization signal that could be observed only at ×1665 magnification (3), predominantly in the endometrial glands (A) and surface epithelium (B) as compared to the stroma (C).

FIG. 14. Analysis of IL-1RII mRNA expression in the endometrium by RT-PCR. RT-PCR was used to assess IL-1RII mRNA expression in the endometrium. A) Graphical illustration of IL-1RII mRNA relative levels (% of control ±SEM) in the endometrium throughout each day of the menstrual cycle. B) Graphical illustration of IL-1RII mRNA relative levels (% of control ±SEM) in the endometrium throughout each phase of the menstrual cycle. C) Representative Southern blots of IL-1RII and GAPDH (internal control) transcripts in the whole endometrial tissue after RT-PCR. 1, Positive control (cDNA preparation from human endometrial tissue expressing IL-1RII); 2, negative control (PCR in the absence of cDNA). Tissues were at Days (d) 10, 13, 17, 21, and 24 of the menstrual cycle. Note the elevated levels of IL-1RII mRNA were observed in endometrial tissues at Day 13 (late proliferative phase) and Day 24 (late secretory phase) of the menstrual cycle and the decreased levels at Day 21 (midsecretory phase). D) Representative Southern blots of IL-1RII and GAPDH from separated stromal (S) and glandular epithelial (E) cells at different days (10, 14, 17, and 27) of the menstrual cycle.

FIG. 15. Western blot analysis of MIF expression in endometriotic tissue. Total proteins were subjected to SDS-PAGE analysis and Western blotting using an affinity purified polyclonal goat anti-MIF antibody (lanes 1-3) or an equivalent concentration of normal goat Ig instead of the primary (lanes 4-6). Lanes 1 and 4, 10 μg total proteins; lanes 2 and 5, 20 μg total proteins; lanes 3 and 6, 40 μg total proteins. The detected band has an estimated apparent molecular weight of approximately 12.5 kDa.

FIG. 16. RT-PCR and Southern blot analysis of MIF transcripts in the endometriotic tissue. Total RNA obtained from endometriotic tissue was reverse transcribed, amplified with MIF (upper lanes) or GAPDH (lower lanes) primers, and hybridized with ³²P-labeled corresponding probes. Lane 1, positive control (cDNA preparation from the human hystiocytic cell line U937, known to secrete MIF); lane 2, negative control (PCR in the absence of cDNA); lanes 3-5, linearity test with different RT volumes.

FIG. 17. Immunohistochemical analysis of MIF expression in endometriotic tissue. The biopsy of a red papular endometriotic lesion from a 37-yr-old woman with stage I endometriosis was submitted to an immunohistochemical analysis. Note the intense brownish immunostaining in the glands and cell aggregates throughout the stroma in the presence of a goat polyclonal anti-MIF antibody (A) and the absence of such staining in the presence of goat IgGs used at concentration equivalent to that of the primary antibody (B) (negative control). Scale bar=20 μm.

FIG. 18. Dual-immunofluorescent staining of MIF and CD3, CD68 or vWF in endometriotic tissue. Tissue sections were incubated with goat polyclonal anti-MIF antibody and with mouse monoclonal anti-CD3, mouse monoclonal anti-CD68, or rabbit polyclonal anti-vWF antibody; sections were then incubated simultaneously with rhodamine-conjugated sheep antimouse antibody and fluorescein isothiocyanate-conjugated donkey antigoat antibody to detect coexpression of MIF with CD3 or CD68 or with rhodamine-conjugated mouse antirabbit antibody and fluorescein isothiocyanate-conjugated donkey antigoat antibody to detect coexpression of MIF with vWF. Dual-immunofluorescent staining of MIF (A-C) and CD3 (D), CD68 (E) or vWF (F) in endometriotic tissue. Note the expression of MIF (green) in CD3-, CD68-, and vWF-positive T lymphocytes, macrophages, and endothelial cells, respectively (red). Superposition of fluorescein (green) and rhodamine (red) signals clearly shows coexpression (yellow signal) of MIF with CD3 (G=A+D), CD68 (H=B+E), and vWF (I=C+F). Scale bars=20 μm.

FIG. 19. Graphical illustration of MIF concentrations as measured by ELISA in endometriotic tissue. Endometriotic biopsies were classified according to their appearance at laparoscopy (red, n=11; typical, n=6; white, n=7) (A) or to endometriosis stage (stage I, n=12; stage II, n=9; stage III-IV, n=3) (B). The box-and-whisker plot was used to illustrate the distribution of MIF concentrations. The box delimits values falling between the 25th and the 75th percentiles and the horizontal line within the box refers to the median scores. *, Significant difference between endometriosis stages I and II using the unpaired t test (P<0.05), **, Significant difference with the red lesions (P<0.01).

FIG. 20. Semiquantitative RT-PCR analysis of MIF mRNA in endometriotic tissue. The quantity of the PCR products was determined by densitometric analysis of the intensity of the hybridization signal, the relative level of MIF mRNA normalized to GAPDH mRNA was calculated, and the results were expressed as percent of control (positive control). A) Types of endometriotic lesions (red, n=9; typical, n=6; white, n=6); B) stage of endometriosis (stage I, n=10; stage II, n=7; stage III-IV, n=3). The box-and-whisker plot was used to illustrate the distribution of MIF mRNA levels. The box delimits values falling between the 25th and the 75th percentiles and the horizontal line within the box refers to the median scores. *, Significant difference with the red lesions (P<0.05).

FIG. 21. IL-1RII mRNA expression in situ in the endometrium of normal women and women with endometriosis. Sections were hybridized with biotin-labeled cDNA probes. Biotin detection was performed using a rabbit polyclonal antibiotin antibody, a biotinylated goat anti-rabbit polyclonal antibody, and fluorescein isothiocyanate-conjugated streptavidin, respectively. Propidium iodine was used to make the nucleus visible in yellow-orange on ultraviolet excitation. Illustration of IL-1RII mRNA expression in situ in the endometrium of normal women (A) and women with endometriosis(B). Note the appearance of endometrial glands (g) and stroma (s) at ×666 magnification (A1 and B1). The hybridization signals (green-yellow; arrow) were visible at 1665× magnification (A2 and B2). Note the greater number of hybridization signals in a part of an endometrial gland from a normal woman (Day 13; A2) compared to that from a woman with stage I endometriosis

FIG. 22. Graphical illustration of IL-1RII mRNA hybridization scores in the endometrium of normal controls and of women with endometriosis of different stages. IL-1RIT mRNA hybridization scores were measured and compared between the endometrium of normal controls (n=26) and the endometrium of women with endometriosis of different stages (n=53). A) Hybridization scores (mean±SEM) in the glands. B) Hybridization scores (mean±SEM) in the stromal compartment. * P<0.05, ** P<0.01.

FIG. 23. RT-PCR and Southern blot analysis of IL-1RII transcripts in the endometrial tissue. Total RNA samples were extracted from endometrial biopsy samples of women with (n=10) and of women without (n=8) endometriosis, then reverse transcribed, amplified with IL-1RII or GAPDH primers, and hybridized with ³²P-labeled corresponding probes. A) Representative Southern blot analysis. Lanes 1 and 2: women with stage I endometriosis, Days 13 and 18 of the menstrual cycle, respectively; lanes 3 and 4: normal women, Days 12 and 16 of the menstrual cycle, respectively. The GAPDH was used as a control. B) The “box and whisker” plot was used to illustrate IL-1RII mRNA levels using semiquantitative RT-PCR. The box delimits values falling between the 25th and the 75th percentiles, and the horizontal line within the box refers to the median scores. **Significant difference between the endometriosis and control groups (P<0.01).

FIG. 24. Soluble IL-1RII concentrations in the serum of control subjects (C) and women with endometriosis stages I-II and III-IV (EIII-IV). The “box and whisker” plot was used to illustrate the distribution of sIL-1RII values between control subjects and women with endometriosis. The box delimits values falling between the 25th and the 75th percentiles and the horizontal line within the box refers to the median. * P<0.01 as compared to control subjects using the unpaired t test.

FIG. 25. IL-1α and IL-1β concentrations in the serum of controls and women with different endometriosis (EI-II and EIII-IV) stages. IL-1α and IL-1β concentrations were measured in the serum of controls and women with different endometriosis (EI-II and EIII-IV) stages. A) Serum concentration of IL-1α in control women (C) and women with different endometriosis (EI-II and EIII-IV) stages. B) Serum concentration of IL-1β in control women (C) and women with different endometriosis (EI-II and EIII-IV) stages. Comparison of control and endometriosis groups was performed with the unpaired t test.

FIG. 26. Serum-induced MCP-1 secretion by U937 cells. 10⁶ cells/well in 24-well culture plates were exposed for 24 hours at 37° C. serum from normal controls and women with endometriosis (5% v/v dilution in FBS-free RPMI medium) and MCP-1 secretion (μg/ml) was measured by ELISA in the culture supernatant. A) controls (C) versus endometriosis (E) patients; B) controls (C) versus endometriosis stages I-II (EI-II) and III-IV (EIII-IV). Data were presented as mean±SEM and analyzed with the unpaired t test, which compares the control group to each endometriosis group. * P<0.05; ** P<0.01.

FIG. 27. Effect of rIL-1RII on serum-induced MCP-1 secretion. The effects of rIL-1RII on serum-induced MCP-1 secretion was measured. Data is presented as mean±SEM and analyzed with the paired t test, which compares sera treated with human rIL-1RII to untreated sera in each group. C=controls; EI-II=endometriosis stages I and II; EIII-IV=endometriosis stages III-IV. * P<0.05; ** P<0.01.

FIG. 28. Effect of human rIL-1ra (100 ng/ml) on serum-induced MCP-1 secretion. The effect of human rIL-1ra on serum-induced MCP-1 secretion was measured. Data is presented as mean±SEM and analyzed with the paired t test, which compares IL-1ra-treated to untreated sera within each group. C=controls; EI-II=endometriosis stages I and II; EIII-IV=endometriosis stages III-IV.

FIG. 29. Effect of human rIL-1β on serum-induced MCP-1 secretion. The effect of human rIL-1β on serum-induced MCP-1 secretion was measured. Data is presented as mean±SEM and analyzed with the paired t test, which compares IL-1β-treated to untreated sera within each group. C controls; E-endometriosis.

FIG. 30. MIF concentrations in the endometrial tissue according to the stage of the disease. MIF concentrations were measured in the endometrial tissue of normal controls (n=25) and patients with endometriosis (n=45) according to the stage of the disease. The distribution of MIF concentrations in the endometrial tissue of normal controls and patients with endometriosis according to the stage of the disease was then plotted. The horizontal lines represent the mean for each set of data.

FIG. 31. MIF concentrations in the endometrial tissue according to the fertility status. MIF concentrations were measured in the endometrial tissue of normal controls (n=25) and patients with endometriosis (n=45) according to their fertility status. A distribution of MIF concentrations in the endometrial tissue of normal controls and patients with endometriosis according to their fertility status was then plotted. The horizontal lines represent the mean for each set of data.

FIG. 32. MIF concentration in the endometrial tissue throughout the menstrual cycle. MIF concentrations were measured in the endometrial tissue of normal women (n=55) throughout the menstrual cycle.

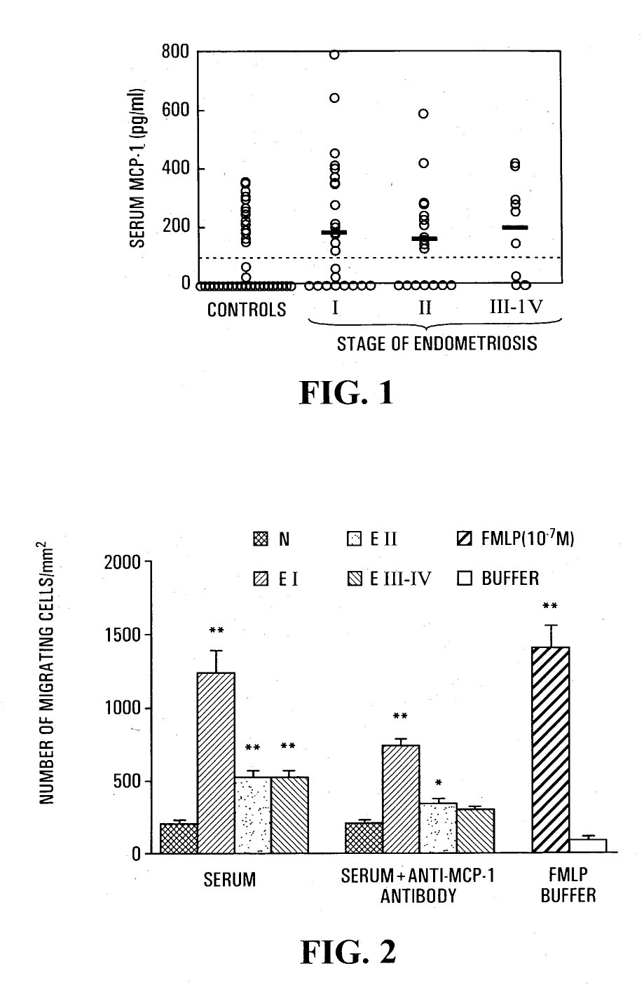

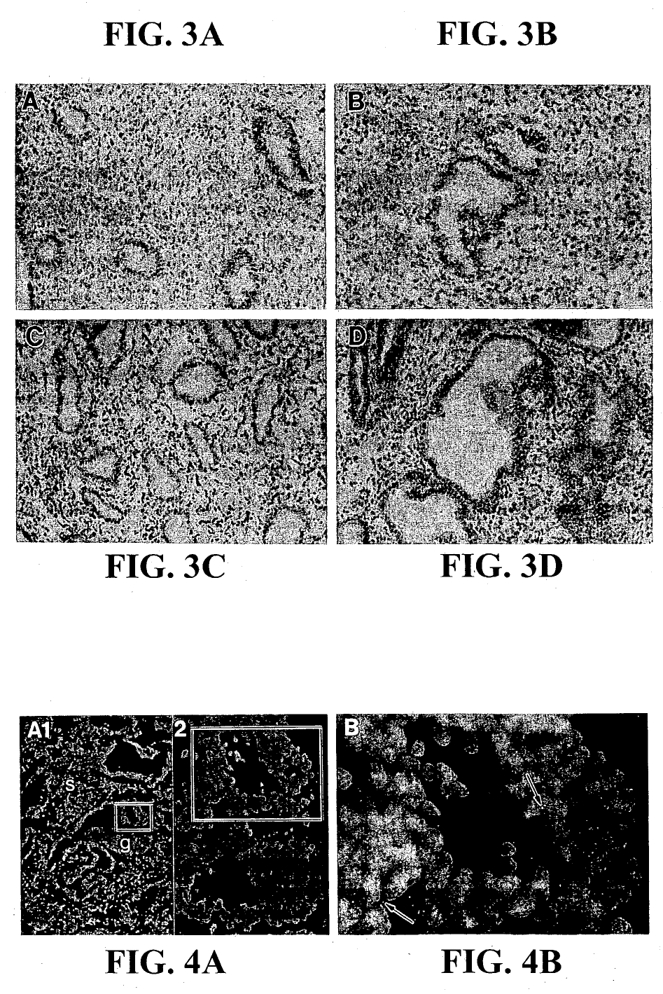

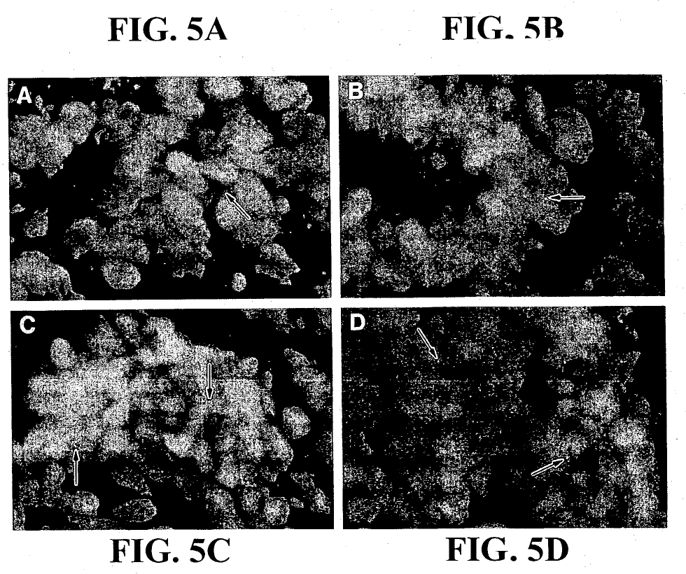

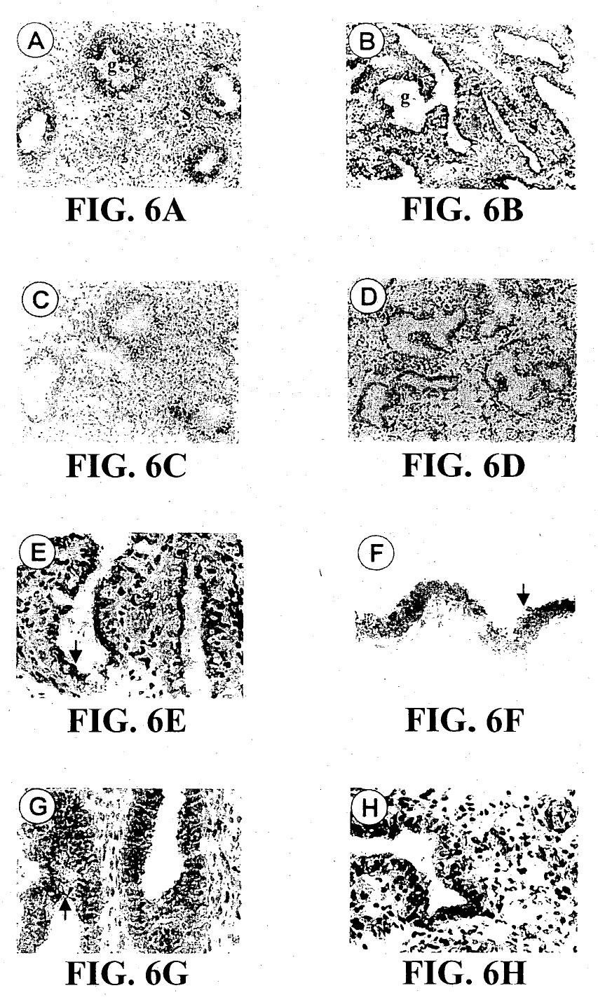

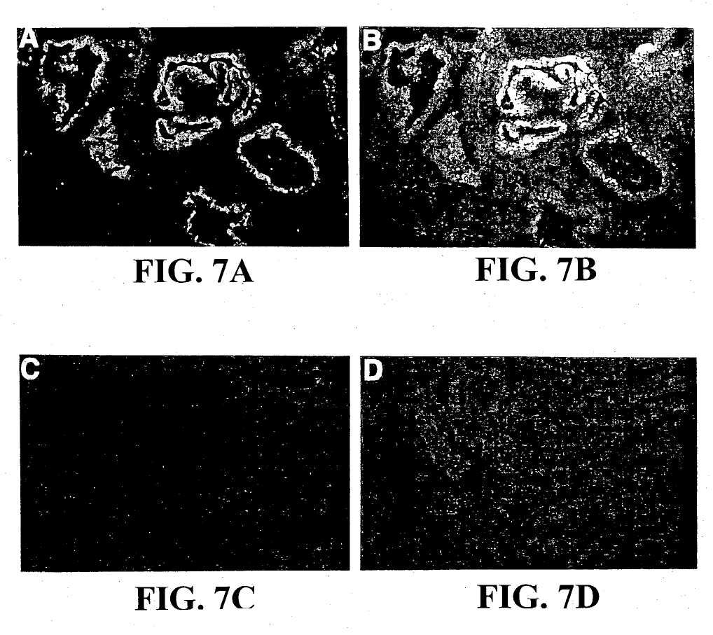

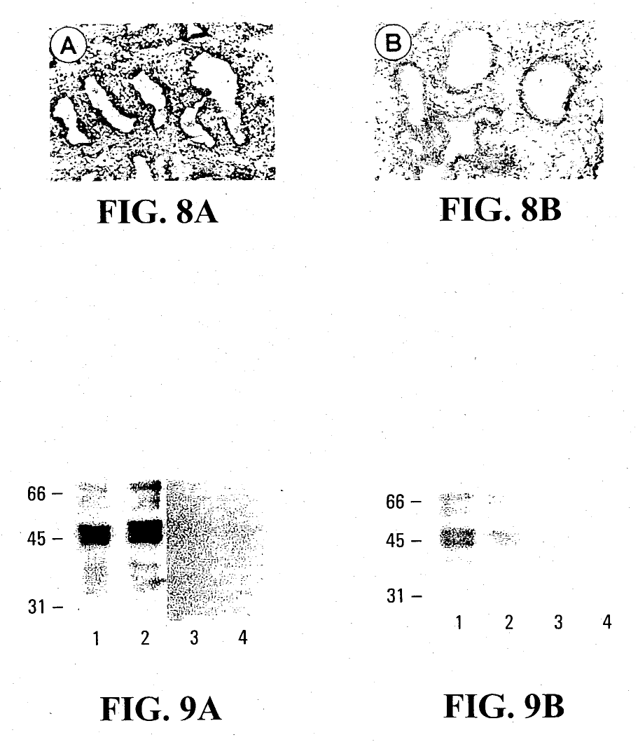

### DETAILED DESCRIPTION OF THE INVENTION

The studies described herein have investigated the relationship between IL-1RII, macrophage inhibitory factor (MIF) and reproductive processes and disease, especially endometriosis and fertility.

MIF is a pro-inflammatory cytokine that also posesses pro-angiogenic properties. MIF has the ability to restrain random migration of macrophages. MIF has been detected in the endometrium of healthy women, in the placenta and in early pregnancy endometrium (Arcuri F et al, 1999; Arcuri F et al, 2001), as well as in the media of cultured endometriotic cells (Yang Y, et al, 2000). MIF has been described as an important modulator for a variety of cell functions, including inflammatory and immune responses (Metz C N, et al, 1997; Nishihira J, et al, 1999), tumor growth-associated angiogenesis in vivo, and autocrine regulation of endothelial cell proliferation in vitro (Chesney J, et al, 1999; Ogawa H, et al, 2000; Shimizu T, et al, 1999). Prior to the results described herein, MIF levels in the endometrium have been shown to be constant throughout the different phases of the menstrual cycle (Arcuri F et al, 1999).

It is shown herein that MIF concentration in endometrial tissues collected from women suffering from endometriosis are higher than from endometrial tissues of healthy women. In addition, MIF was shown to be highly expressed in (e.g. flame-like red) endometriotic lesions. These results demonstrate a role for MIF in the onset of endometriosis and the persistence of endometriotic lesions. Suprinsingly, MIF concentration in the endometrium is further elevated in the later stages of endometriosis and is associated with infertility. As such, levels of MIF are correlated with endometriosis disease progression and infertility. The newly discovered role of MIF in endometrial tissue and associated conditions thus offers novel strategies for diagnostic, prognostic and predictive methods concerning reproduction-associated diseases.

In the results presented herein, it was found that MIF was effectively expressed by ectopic endometrial tissue, both at the mRNA and protein levels, as assessed by RT-PCR and Western blotting. It is shown herein that MIF was located in the glands as well as in cell aggregates scattered over the stroma. Dual-immunofluorescence analysis identified endothelial cells, macrophages, and T lymphocytes as cells markedly expressing MIF in the stroma. MIF was found to be highly produced in the endometriotic lesions that were presenting noticeable vascularization and leukocyte infiltration. The findings disclosed herein clearly demonstrate that MIF can be produced locally in endometriotic tissue, and by different cell types. Endometriotic lesions can be classified according to their appearance and to their activity. In fact, lesions of the peritoneal lining of the pelvis have various macroscopic appearances, which reflect their age and/or activity. The red subtle lesions are more vascularized and have a higher epithelial mitotic index than the typical, puckered black or bluish peritoneal lesions, whereas the vascularization and the mitotic index are lower in the white lesions. Thus, red lesions are thought to correspond to the first, active stage of early implantation of endometrial glands and stroma and would evolve toward the typical black or bluish lesion after enclosure beneath the peritoneal lining. The white lesions, which are believed to correspond to fibrotic quiescent lesions, show less vascularization and/or mitotic activity and represent less active forms of the disease (Wiegerinck M A, et al, 1993; Kokorine I, et al, 1997; Nisolle M, et al, 1993; Nisolle M, et al, 1997). The results presented herein reveal that MIF expression is significantly higher in red subtle than in typical black/blue or white endometriotic lesions. The protein expression, as measured by ELISA, according to endometriotic lesion type was in keeping with that of mRNA as assessed by semiquantitative RT-PCR. The results suggest that the higher expression of MIF in the red lesions might reflect its role in promoting/maintaining a higher degree of vascular development and support that MIF plays an important role in endometriosis-associated active angiogenesis and inflammatory processes and as a marker of active disease. The results presented herein also show that MIF was more markedly expressed in lesions from the initial stage of endometriosis (stage I), compared with the more advanced stages, which makes plausible the involvement of this factor in the initial steps of endometriotic tissue growth and development.

In the studies described herein, MIF expression has also been characterized in the endometrium of healthy women during different days and phases of the menstrual cycle. Unexpectedely and contrary to previous reports (Arcuri F et al, 2001), low levels of MIF are shown herein to correlate with the implantation window of the fertilized egg, thus providing MIF-based methods of determining endometrial receptivity and fertility and for the determination of the implantation window.

The results presented therein suggest that MIF may have multiple functions in human endometrium and that its secretion is subjected to subtle chronological regulation. This is remarkable as the phase of reduced expression correlates to a putative “implantation window” thought to exist within the mid-secretory phase between days 18 and 22 (Tabibzadeh S et al., 1998). The findings point out that the relative decrease in MIF expression at that specific time of the cycle where embryonic attachment and implantation may occur is suggestive of a possible role for MIF in the initial interactions between maternal and embryonic cells and the establishment of an endometrial period of receptivity.

Interleukin-1 (IL-1) is a pro-inflammatory cytokine that acts in the endometrium. IL-1 type II receptor (IL-1RII) is a “decoy” receptor for IL-1. IL-1RII can bind IL-1 and inhibit IL-1 specific signaling through IL-R type I receptor. In other words, IL-1RII is a specific natural inhibitor of the cytokine IL-1. Endometrial IL-1RII expression is also downregulated in the endometrium of women suffering from endometriosis.

The studies described herein show that significantly lower levels of IL-1RII were detected in the endometrial tissues of women affected with endometriosis compared to tissues from healthy women. In addition, lower IL-1RII levels have been detected in the endometrial tissues collected from women in the earliest stages of endometriosis (Stages I-II). These results show a correlation of reduced levels of IL-1RII with the more pronounced inflammation in the endometrium of women affected by endometriosis. In addition, since IL-1RII levels are further decreased in the early stages of the disease, a link of IL-1RII levels to the onset of endometriosis is further demonstrated. Surprisingly, a difference was observed in the serum levels of IL-1RII between healthy women and women suffering from endometriosis. No difference was observed between the IL-1 serum level of the two groups. These results demonstrate that reduced expression of Il-1RII is an early marker of endometriosis, and that the serum levels of IL-1RII unexpectedly reflect endometrial IL-1RII, thus providing more clinically amenable methods of diagnosis and prognostication of reproduction-associated diseases.

In the studies described herein, the role of IL-1RII-in endometriosis was further investigated. The serum of healthy women was added to the culture medium of the hystiocytic cell line U937, its ability to induce monocyte chemotactic protein 1 (MCP-1) was evaluated and compared to the serum collected from women suffering from endometriosis. MCP-1 is another pro-inflammatory cytokine that has been shown earlier to be linked to endometriosis. Suprisingly, it was shown that the serum derived from women suffering from endometriosis elicited a-higher secretion of MCP-1 from U397 cells than the serum derived from healthy women. The addition of exogenous IL-1RII to the serum of affected women decreased MCP-1 secretion from U937 cells. The addition of recombinant IL-1ra or IL-1 to the different pools of serum did not significantly modulate the secretion of MCP-1 by U937 cells. These results demonstrate that the decrease in IL-1RII seen in the serum of women affected by endometriosis plays a role in disease onset, and that restoring or increasing IL-1RII levels represents a strategy for the treatment of reproduction-associated disease.

As described herein, circulating sIL-1RII was measured in the peripheral blood of normal and endometriosis women, and a significant decrease in its levels was found in women having the disease. It was shown that sIL-1RII levels were significantly reduced in endometriosis stages I-II, both in the proliferative and the secretory phases of the menstrual cycle. These results suggest a deficiency in the regulation of IL-1 action at the systemic level in the initial stages of endometriosis. In further studies, U937 monocytic cells were exposed to sera from normal and endometriosis women, and cell activation was estimated by measuring MCP-1 secretion by these cells. The results described herein clearly show that the U937 monocytic cells produced larger amounts of MCP-1 in response to peripheral blood sera from women with endometriosis. They further revealed that such a serum-induced MCP-1 secretion occurred in endometriosis stages I-II, and that sera from women with more advanced endometriosis stages (III-IV) had no other significant effect. To investigate whether the reduced levels of sIL-1RII observed in the peripheral blood of women with endometriosis may account for the increased reactivity of U937 cells towards endometriosis-women derived sera, human rIL-1RII was added to the different pools of serum prior to incubation with U937 cells. Interestingly, it was shown that rIL-1RII significantly reduced MCP-1 production by U937 cells in response to serum from normal and endometriosis women. However, the most significant decrease was observed in women with endometriosis stages I and II, which is in keeping with the findings herein showing reduced levels of sIL-1RII (soluble IL-1RII) in the peripheral blood of these patients.

In the studies described herein, IL-1RII (protein and mRNA) expression was analyzed according to the day or phase of the menstrual cycle. Unexpectedly, it has been found that IL-1RII concentration is lowered during the implantation window, thereby suggesting a role for IL-1RII as a marker for endometrial receptivity. As such, the results described herein further provide methods of prediction of the implantation window based on the levels of IL-1RII as well as IL-1RII-based therapeutic intervention in fertility-associated conditions.

Immunoreactive IL-1RII was detected throughout endometrial tissue both in epithelial and stromal compartments, but it was more obvious in endometrial glands and surface epithelium. The results described herein show that both cellular and extracellular/luminal IL-1RII immunoreactivity varied within the menstrual cycle and followed a regulated cycle phase-dependent pattern. These results show that the pattern of IL-1RII immunostaining in endometrial epithelial cells was quite unusual, and the temporal expression for this natural inhibitor of IL-1 is rather interesting. That the receptor expression is down-regulated in the midsecretory phase, especially during the implantation window, and up-regulated at the end of the menstrual cycle suggests that IL-1RII may have multiple functions in human endometrium. Epithelial IL-1RII expression and distribution in endometrial tissue across the menstrual cycle is remarkable for several reasons. First, IL-1RII appeared to be expressed and released predominantly by glandular and luminal epithelial cells rather than by stromal cells. This is of additional interest, because first interactions between the embryo and the endometrium occur at the level of the luminal epithelial cells during the adhesion process. Second, the significant decline in IL-1RII luminal staining, which started at the beginning of the secretory phase and reached its minimum at approximately Day 22, in the midsecretory phase, is rather interesting and suggests that IL-1RII secretion is subjected to subtle chronological regulation in endometrial epithelial cells. Third, IL-1RII expression in endometrial tissue markedly increased in the late secretory phase of the menstrual cycle, both at the level of the protein and of the mRNA. This may have a considerable significance, because in the absence of implantation, the endometrial tissue undergoes a process of cell necrosis and disintegration at the end of the menstrual cycle (Tabibzadeh S, 1991).

Therefore, it is shown herein that that levels of IL-1RII and levels of MIF correlate with various characteristics of reproductive tissue, including reproduction-associated diseases and endometrial receptivity/determination of the implantation window. As such, the role of IL-1RII and MIF in endometriosis and infertility may be exploited to develop predictive methods to assess fertility and reproduction-associated diseases.

Therefore, in a first aspect, the invention relates to methods for the determination of MIF levels in a biological sample obtained from a subject for the diagnosis, prognostication, or both, of reproduction-associated diseases. An elevated level of MIF is a marker for the onset and/or progression of reproduction-associated diseases. In one embodiment, the subject is a mammal, in a further embodiment, a human. In another embodiment, the biological sample is an endometrial derivative (i.e. is derived from the endometrium or tissue or cells thereof).

“Reproduction-associated disease” as used herein refers to diseases which affect at least one reproductive process or normal function of reproductive tissue. Such disease include, but are not limited to, endometriosis and infertility.

Thus, aspects of the present invention are methods for the diagnosis and prognostication of reproduction-associated diseases, via assessing a level of a MIF protein (or a protein with an amino acid sequence substantially identical thereto) or a nucleic acid (e.g. an mRNA) encoding MIF (or a nucleic acid substantially identical thereto) or MIF activity. MIF protein may be assessed by various methods known in the art, including but not limited to immunodetection methods such as immunohistological staining of proteins in a sample and immunoassays such as enzyme-linked immunoassay (ELISA), enzyme immunoassay (EIA), radioactive immunoassay (RIA) and Western analysis (immunoblotting). MIF mRNA levels may be assessed by various methods known in the art (Sambrook et al, 1989) such as reverse transcriptase-polymerase chain reaction (RT-PCR), and Northern analysis using an appropriate probe(s). Another example is in situ hybridization where transcripts are detected using a specific probe on a sample. SEQ ID NOs 7 and 8 set forth, for example, DNA and polypeptide sequences of human MIF.

The invention also relates to methods for determining the levels of MIF or IL-1RII, in a biological sample obtained from a subject, to assess endometrial receptivity. MIF and IL-1RII are both downregulated during the implantation window of the fertilized egg and their level is thus indicative of endometrial receptivity. In an embodiment, the levels of the cytokine detected are compared to an established standard, to the levels measured in a control sample or to the levels measured earlier in the same subject. In another embodiment, the subject is in the secretory phase of her menstrual cycle. In an embodiment, the biological sample is a body fluid or tissue, in a further embodiment, the biological sample is derived from endometrial tissue.

“Endometrial receptivity” as used herein refers to the ability of the endometrium to support the implantation of a fertilized egg. “Implantation window” as used herein refers to the period where the endometrium supports the attachment of a fertilized egg and allows its growth. “Secretory phase” as used herein refers to the period, in the menstrual cycle, spanning between the ovulation and the menses.

Thus, further aspects of the present invention are methods for the determination of endometrial receptivity, via assessing a level of a MIF protein or a nucleic acid (e.g. an mRNA) encoding MIF or MIF activity or IL-1RII protein or a nucleic acid (e.g. an mRNA) encoding IL-1RII or IL-1RII activity. MIF and IL-1RII protein levels may be assessed by various methods known in the art, such as the immunodetection-methods noted above. MIF and IL-1RII mRNA levels may similarly be assessed by the above-noted methods.

In addition, the invention relates to methods for the diagnosis, prognostication, or both, of reproduction-associated diseases, based on the determination IL-1RII levels (e.g. in serum) from a subject. A low level of IL-1RII is indicative of reproduction-associated diseases. In one embodiment, the subject is a mammal, in a further embodiment, a human. In another embodiment, the biological sample is an endometrial derivative.

Thus, further aspects of the present invention are methods for the diagnosis and prognostication of reproduction-associated diseases, via assessing a level of a IL-1RII protein or a nucleic acid (e.g. an mRNA) encoding IL-1RII or IL-1RII activity. IL-1RII protein and mRNA levels may be assessed by various methods known in the art, such as those noted above.

The invention relates further to a method for treating reproduction-associated diseases by enhancing/increasing IL-1RII activity or expression in a cell, tissue or subject. In an embodiment, the method comprises administering an effective amount of IL-1RII or IL-1RII related compound to a subject, or a cell or tissue thereof. In an embodiment, IL-1RII expression is upregulated. In one embodiment, the subject is a mammal, in a further embodiment, a human. In another embodiment, the cell and tissue are derived from the endometrium.

With regard to increasing or upregulating expression of a IL-1RII in a cell, various methods of introducing IL-1RII-encoding nucleic acids into the cell may be used, examples of which are described below. Methods such as the gene therapy methods discussed below may be used in this regard. An example of an IL-1RII-encoding nucleic acids is the human IL1-RII DNA sequence set forth in SEQ ID NO:9, a nucleic acid capable of encoding an IL1-RII polypeptide (e.g. the human IL1-RII polypeptide sequence set forth in SEQ ID NO:10), or nucleic acids substantially identical thereto. The method may also comprise administering to an affected area or endometrial tissue a cell comprising such a IL-1RII-encoding nucleic acid, via, for example, transplantation or introduction of a endometrial cell or precursor thereto (e.g. a stem cell) comprising such a IL-1RII-encoding nucleic acid. Further, the method may entail administering to the subject a compound capable of unpregulating or increasing expression of a IL-1R11.

A nucleic acid of the invention may be delivered to cells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson el al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel el al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application Wo 89/02468; PCT Application WO 89/05345; and PCT Application WO 92/07573).

For use as a gene therapy vector, the genome of an adenovirus may be manipulated so that it encodes and expresses a peptide compound of the invention, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin el al. (1992) Proc. Natl. Acad. Sci. USA 89:2581-2584).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260. may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

General methods for gene therapy are known in the art. See for example, U.S. Pat. No. 5,399,346 by Anderson et al. A biocompatible capsule for delivering genetic material is described in PCT Publication WO 95/05452 by Baetge et al. Methods of gene transfer into hematopoietic cells have also previously been reported (see Clapp, D. W., et al., Blood 78: 1132-1139 (1991); Anderson, Science 288:627-9 (2000); and, Cavazzana-Calvo et al., Science 288:669-72 (2000)).

The invention further relates to a commercial package for the assessment of MIF levels in a tissue or body fluid obtained from a subject and instructions for the diagnosis, prognostication, or both, of reproduction-associated disease. In an embodiment, the commercial package further comprises a reference value or control sample. In a further embodiment, the reference value is an established standard of MIF protein, MIF encoding RNA, or MIF activity; and the control sample is a corresponding level of MIF protein, MIF encoding RNA, or MIF activity in a tissue or body fluid of a control subject. In another embodiment, the tissue or body fluid is derived from the endometrium.

The invention further relates to a commercial package for the assessment of MIF and IL-1RII levels in a tissue or body fluid obtained from a subject and instructions for the determination of endometrial receptivity. In an embodiment, the commercial package further comprises a reference value or control/reference sample. In a further embodiment, the reference value is an established standard of MIF protein, IL-1RII protein, MIF encoding RNA, IL-1RII encoding RNA, IL-1RII activy or MIF activity; and the control sample is a corresponding level of of MIF protein, IL-1RII protein, MIF encoding RNA, IL-1RII encoding RNA, IL-1RII activy or MIF activity in a tissue or body fluid of a control subject. In another embodiment, the tissue or body fluid is an endometrial derivative.

The invention further relates to a commercial package for the assessment of IL-1RII levels in a tissue or body fluid obtained from a subject and instructions for the diagnosis, prognostication, or both, of reproduction-associated disease. In an embodiment, the commercial package further comprises a reference value or control sample. In a further embodiment, the reference value is an established standard of IL-1RII protein, IL-1RII encoding RNA, or IL-1RII activity; and the control sample is a corresponding level of IL-1RII protein, IL-1RII encoding RNA, or IL-1RII activity in a tissue or body fluid of a control subject. In another embodiment, the tissue or body fluid is an endometrial derivative.

The invention further relates to a composition for the prevention or treatment of reproductive-associated disease in a subject comprising an effective amount IL-1RII or a IL-1RII-related compound in admixture with a pharmaceutically acceptable carrier.

“IL-1RII-related compound” as used herein refers to a compound which is structurally and/or functionnally related to IL-1RII. Such compounds include homologs, variants or fragments to a IL-1RII protein which retain IL-1RII activity. Such a compound may comprise a peptide which is substantially identical to an IL-1RII protein or fragment thereof. Similarly, such a compound includes a polypeptide or protein encoded by a nucleic acid sequence which is substantially identical to, or is related by hybridization criteria (see below) to a nucleic acid sequence capable of encoding an IL-1RII. Examples of IL1-RII polypeptides and IL-1RII-encoding nucleic acids are set forth below. Such compound include fragments such as a soluble version or domain of an IL1-RII. Such compounds further include precursors or prodrugs which are metabolized or otherwise converted to an active compound at the site of action.

A homolog, variant and/or fragment of a IL-1RII which retains activity may also be used in the methods of the invention. Homologs include protein sequences which are substantially identical to the amino acid sequence of a IL-1RII, sharing significant structural and functional homology with a IL-1RII. Variants include, but are not limited to, proteins or peptides which differ from a IL-1RII by any modifications, and/or amino acid substitutions, deletions or additions. Modifications can occur anywhere including the polypeptide backbone, (i.e. the amino acid sequence), the amino acid side chains and the amino or carboxy termini. Such substitutions, deletions or additions may involve one or more amino acids. Fragments include a fragment or a portion of a IL-1RII or a fragment or a portion of a homolog or variant of a IL-1RII.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides. or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences-may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with IL-1RII protein or a nucleic acid encoding a IL-1RII protein.

Substantially complementary nucleic acids are nucleic acids in which the “complement” of one molecule is substantially identical to the other molecule. Optimal alignment of sequences for comparisons of identity may be conducted using a variety of algorithms, such as the local homology algorithm of Smith and Waterman (Smith et al, 1981), the homology alignment algorithm of Needleman and Wunsch (Needleman et al, 1970), the search for similarity method of Pearson and Lipman (Pearson et al, 1988), and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al (Altschul et al, 1990; using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff et al, 1992) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% sodium dodecyl sulfate (SDS), 1 mM EDTA at 65° C., and washing in 0.2×SSC/0.1% SDS at 42° C. (Ausubel et al 1989). Alternatively, hybridization to filter-bound sequences under stringent conditions may, for example, be performed in 0.5 M NaHPO₄, 7% SDS, 1 mM EDTA at 65° C., and washing in 0.1×SSC/0.1% SDS at 68° C. (Ausubel et al, 1989). Hybridization conditions may be modified in accordance with known methods depending on the sequence of interest (Tijssen, 1993). Generally, stringent conditions are selected to be about 5° C. lower than the thermal melting point for the specific sequence at a defined ionic strength and pH.

As noted above, the invention further relates to therapeutic methods comprising administering a therapeutically effective amount of an IL1-RII or IL1-RII related compound to a subject in need thereof.

A “therapeutically effective amount” refers to an amount effective, at dosages and for periods of time necessary, to achieve the desired therapeutic result, such as reduction of reproduction-associated disease or reproduction-associated disease progression. A therapeutically effective amount of a IL-1RII, or a IL-1RII related compound, may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting neural or neurodegenerative disease onset or progression. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, monostearate salts and gelatin. Moreover, the IL-1RII or a IL-1RII-related compound, can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound (e.g. a IL-1RII or a IL-1RII-related compound) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. In accordance with an alternative aspect of the invention, a IL-1RII, or a IL-1RII-related compound, may be formulated with one or more additional compounds that enhance its solubility.

The invention relates to a composition for enhancing endometrial receptivity in a subject, said composition comprising a IL-1RII inhibitor in admixture with a pharmaceutically acceptable carrier. MHR—what do you think, in light of our earlier discussion?—we should probably remove it.

The invention further relates to a composition for lowering endometrial receptivity in a subject, said composition comprising a IL-1RII or a IL-1RII related compound in admixture with a pharmaceutically acceptable carrier.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

Throughout this application, various references are referred to describe more fully the state of the art to which this invention pertains. The disclosures of these references are hereby incorporated by reference into the present disclosure.

### EXAMPLES

### Example 1

### Immunohistochemical Analysis of IL-1RII Protein Expression in Endometrial Tissues of Healthy Women

Subjects. Women who participated in the study provided informed consent for a protocol approved by the Saint-Francois d'Assise Hospital Ethics Committee on Human Research. These women were aged between 23 and 47 yr (mean±SD, 34.6±5.0 yr). They were fertile, requested tubal ligation, and had a normal and regular menstrual cycle. None had visible endometrial hyperplasia or neoplasia, inflammatory disease, or endometriosis at the time of clinical examination or laparoscopy. Women were not receiving any anti-inflammatory or hormonal medication at least 3 mo before laparoscopy. The cycle day was determined according to the cycle history and histological criteria (Noyes R W, et al, 1995). Eighteen women were in the proliferative phase and 24 in the secretory phase.

Collection of endometrial biopsy specimens. Endometrial biopsy specimens were obtained using sterile pipelle (Unimar, Inc., Neuilly-en-Thelle, France). Samples were placed at 48° C. in sterile Hank balanced salt solution (HBSS; Gibco BRL, Burlington, ON, Canada) containing 100 U/ml of penicillin, 100 mg/ml of streptomycin, and 0.25 mg/ml of amphotericin. Samples were then immediately transported to the laboratory, washed twice in HBSS at 48° C., then snap-frozen on dry ice and kept at −80° C. in Eppendorf tubes for Western blot and reverse transcription-polymerase chain reaction (RT-PCR) analyses or in Tissue-Tek OCT compound (Miles, Inc., Elkhart, Ind.) for immunohistochemical studies.

Serial 4-μm cryosections were placed on poly-L-lysine-coated glass microscope slides and fixed for 20 min in formaldehyde (4% [v/v] in PBS; Fisher Scientific, Montreal, PQ, Canada). All incubations were performed at room temperature in a humidified chamber. Sections were rinsed in PBS, immersed in PBS/1% (v/v) Triton X-100 for 20 min at room temperature, rinsed again in PBS, and then treated for 20 min with hydrogen peroxide (H₂O₂, 0.3% [v/v] in absolute methanol) to eliminate endogenous peroxidase. After a PBS rinse, immunostaining was performed using a mouse monoclonal anti-human IL-1RII antibody (primary antibody; R&D Systems, Minneapolis, Minn.) at 15 μg/ml in PBS containing 1% (w/v) BSA with a Vectastain Elite ABC kit (Vector Laboratories, Burlingame, Calif.) and diaminobenzidine (Sigma Chemical Co., St. Louis, Mo.) as chromogen.

Sections incubated without the primary antibody or with nonimmune mouse serum were included as negative controls in all experiments. Slides were viewed using a Leica microscope (model DMRB; Leica Mikroskopie und Systeme GmbH, Postfach, Wetzlar, Germany), and photomicrographs were taken with Kodak 100 ASA film (Kodak, Toronto, ON, Canada). The IL-1RII immunostaining was evaluated in a blinded fashion by two independent observers having no knowledge of laparoscopic findings. The intensity of staining was evaluated three times in three different areas that were randomly selected in the section, and a mean score was given using an arbitrary scale (0=absent, 1=light; 2=moderate, and 3=intense). High concordance between the two observers was found as determined by the kappa (κ) measure of agreement (κ=0.89) (Armitage P, et al, 1994).

Statistical Analyses. The IL-1RII immunostaining scores followed an ordinal scale. Therefore, statistical analyses were performed using nonparametric methods. The association between immunostaining scores and the periods of IL-1RII expression in the menstrual cycle as well as intergroup comparisons of immunostaining scores were analyzed using the Fisher exact test, and the Bonferroni procedure was applied when more than two groups were compared. The correlation between the day of menstrual cycle and the immunohistochemistry scores was evaluated using the Spearman correlation coefficient. The threshold days between the different levels of staining (0, 1, 2, and 3) were determined using the best combination of sensitivity and specificity values for a series of cut-off days within the menstrual cycle. These threshold days allowed us to define or to delimit different expression periods. All analyses were performed using Statistical Analysis System (SAS Institute, Inc., Cary, N.C.). Differences were considered to be statistically significant at P<0.05.

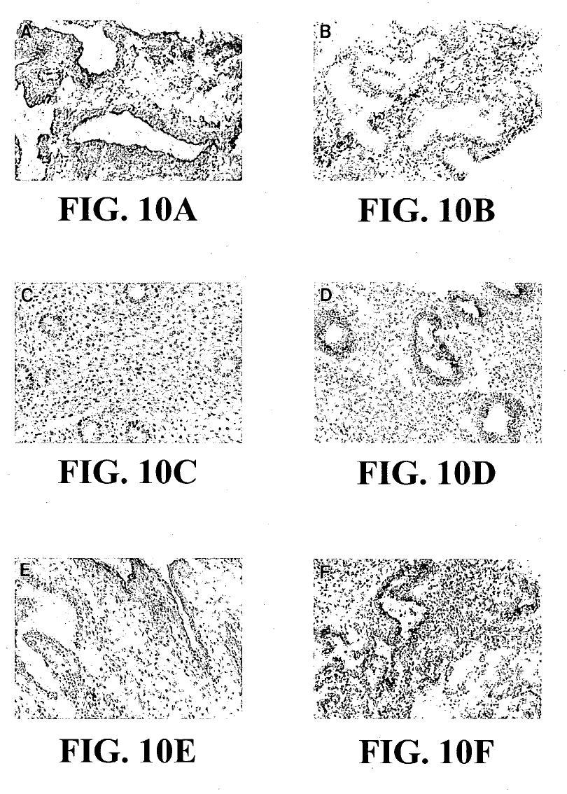

A monoclonal antibody was used to detect IL-1RII protein in endometrial tissue sections. Different concentrations of the antibody (5, 10, 15, and 20 μg/ml) were tested to determine the optimal concentration to use. This experiment was performed on three different series of biopsy specimens from different phases of the menstrual cycle. A concentration of 15 μg/ml was selected, because it allowed an optimal ratio of specificity (minimal background) and sensitivity (detectable positive signal). Examples of positive immunostaining with anti-IL-1RII antibody are shown in FIG. 10A. Immunoglobulins of the same isotype and species, when used at an equivalent concentration as that of the antibody (15 mg/ml), did not display any detectable immunoreactivity (FIG. 10B). Immunoreactive IL-1RII was detectable throughout endometrial tissue, both in the stroma and the glands. Brown immunostaining could be seen around cells (cellular staining) and along the apical border of luminal and glandular epithelium (luminal staining). This was also observed, although less markedly, in microvessels and isolated aggregates throughout the stroma in sections from late secretory endometrial tissues (FIG. 10A). It is noteworthy that luminal secretion was not uniform for all endometrial sections examined.

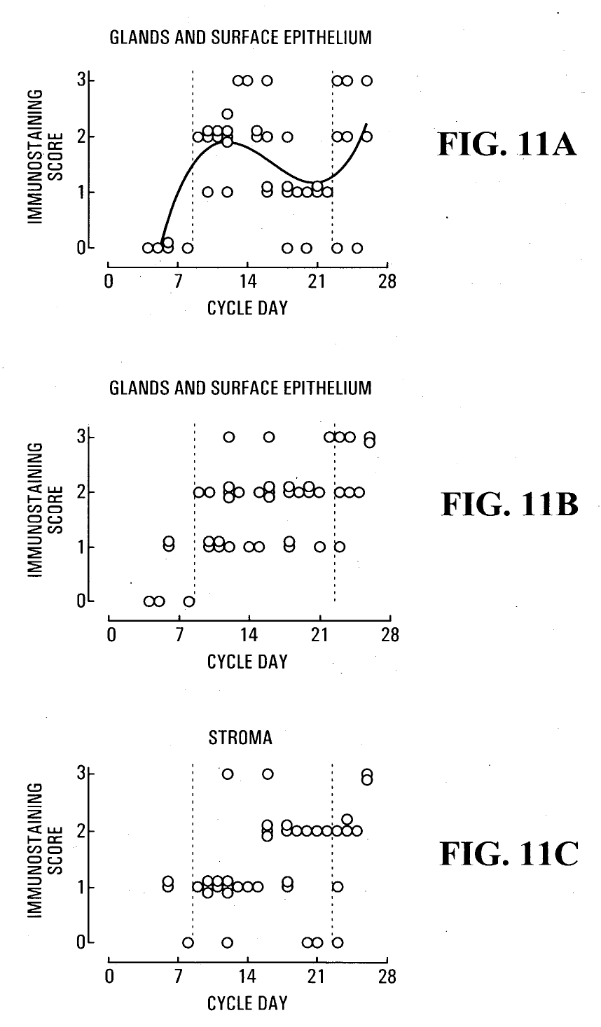

The IL-1RII immunostaining was assessed semiquantitatively by two independent observers in a double-blind manner, taking into account the intensity as well as the distribution of the immunostaining as described above. Cellular and extracellular staining were scored independently in the stroma and in the glands and surface epithelium. Score distributions according to the day of the menstrual cycle are shown graphically in FIG. 11.

To better understand IL-1RII cyclic variations, expression was defined throughout the menstrual cycle using the best combination of sensitivity and specificity values for different cut-off days within the cycle. Our analysis revealed that after Day 8, both cellular (sensitivity 100.0%, specificity=94.9%, P<0.001) and luminal (sensitivity=55.6%, specificity=100.0%, P<0.001) immunostaining became significantly detectable in epithelial cells. A further increase in the intensity of staining occurred after Days 21 and 22, but this increase was more obvious at the cellular (Day 21, sensitivity=88.6%, specificity=71.4%, P<0.01) than at the luminal (Day 22, sensitivity=86.1%, specificity=50.0%, P=0.07) level. In stromal cells, cellular staining remained weak to absent throughout the whole proliferative phase of the menstrual cycle, but it increased significantly after Day 15 at the beginning of the secretory phase (P<0.001). Extracellular staining was virtually undetectable in the stroma, except weakly after Day 21 in tissues from mid to late secretory endometria (sensitivity=91.2%, specificity=85.7%, P<0.001).

Statistical analysis of the data, using the Spearman correlation coefficient, revealed a significant, positive correlation between cellular staining scores and day of the menstrual cycle, both in epithelial (R=0.59, P<0.001) and stromal (R=0.46, P<0.01) cells. However, no positive correlation between epithelial luminal staining and day of the menstrual cycle was found (R=0.17, P=0.29), most probably because of a more fluctuating expression pattern. To better delineate these relationships, we determined the mean values of immunostaining scores for each cycle day, and we found that they follow a third-order polynomial curve (Y=A+BX+CX2+DX3) (FIG. 11). This curve shows that after a maximal increase at approximately Day 12 in the proliferative phase of the menstrual cycle, luminal staining of IL-1RII declined gradually in the secretory phase, reaching its minimal level at approximately Day 22 before increasing again progressively until the end of the cycle. The midsecretory drop in the intensity of IL-1RII luminal immunostaining (Days 19-22) was statistically significant as compared to late proliferative/early secretory (Days 9-18; P<0.05) and late secretory (Days 23-28; P<0.01) immunostaining levels.

Representative examples of IL-1RII immunostaining in the endometrium throughout the menstrual cycle are shown in FIG. 10 for Days 6 (FIG. 10C), 13 (FIG. 10D), 18 (FIG. 10E), and 23 (FIG. 10F). Note the reduction of IL-1RII luminal secretion in the glands and surface epithelium of specimens at Day 18 as compared to Days 13 and 23.

### Example 2

### Western Blot Analysis of ILL-RII Protein Expression in the Endometrium of Healthy Women

Subjects. Women who participated in the study provided informed consent for a protocol approved by the Saint-Francois d'Assise Hospital Ethics Committee on Human Research. These women were aged between 23 and 47 yr (mean±SD, 34.6±5.0 yr). They were fertile, requested tubal ligation, and had a normal and regular menstrual cycle. None had visible endometrial hyperplasia or neoplasia, inflammatory disease, or endometriosis at the time of clinical examination or laparoscopy. Women were not receiving any anti-inflammatory or hormonal medication at least 3 mo before laparoscopy. The cycle day was determined according to the cycle history and histological criteria (Noyes R W, et al, 1995). Eighteen women were in the proliferative phase and 24 in the secretory phase.

Collection of endometrial biopsy specimens. Endometrial biopsy specimens were obtained using sterile pipelle (Unimar, Inc., Neuilly-en-Thelle., France). Samples were placed at 48° C. in sterile Hank balanced salt solution (HBSS; Gibco BRL, Burlington, ON, Canada) containing 100 U/ml of penicillin, 100 mg/ml of streptomycin, and 0.25 mg/ml of amphotericin. Samples were then immediately transported to the laboratory, washed twice in HBSS at 48° C., then snap-frozen on dry ice and kept at −80° C. in Eppendorf tubes for Western blot and reverse transcription-polymerase chain reaction (RT-PCR) analyses or in Tissue-Tek OCT compound (Miles, Inc., Elkhart, Ind.) for immunohistochemical studies.

Frozen endometrial tissues were directly homogenized with the use of a microscale tissue grinder (Kontes, Vineland, N.J.) in a buffer containing 0.5% (v/v) Triton X-100, 10 mM Hepes (pH 7.4), 150 mM NaCl, 2 mM EGTA, 2 mM EDTA, 0.02% (w/v) NaN₃ (Sheth K V, et al, 1991), and a mixture of antiproteases composed of 5 μM aprotinin, 63 μM leupeptin, and 3 mM PMSF. Tissue homogenate was then incubated at 48° C. for 45 min under gentle shaking and centrifuged at 11 000 g for 30 min to recover the soluble extract. Total protein concentration was determined using the Bio-Rad DC Protein Assay (Bio-Rad Laboratories Ltd., Mississauga, ON, Canada). One-hundred micrograms of protein from each extract were heated in a boiling bath for 5 min in 5×SDS sample buffer (1.25 M Tris-HCl [pH 6.8], 50% [v/v] glycerol, 25% β-mercaptoethanol, 10% [w/v] SDS, and 0.01% [w/v] bromophenol blue), separated by SDS-PAGE in 10% (w/v) acrylamide linear-gradient slab gels, and transferred onto 0.45-μm nitrocellulose membranes (Schleicher & Schuell, Keene, NH) using an electrophoretic transfer cell (Bio-Rad). Equal loading in each lane was confirmed by staining the blots with Ponceau S (2% [w/v]). Nitrocellulose membranes were then immersed in PBS containing 5% (w/v) skimmed milk and 0.1% (v/v) Tween 20 (blocking solution) for 1 h at 37° C., cut into strips, and incubated overnight at 48° C. with a monoclonal mouse antihuman IL-1RII antibody (2 μg/ml of blocking solution; R&D Systems) or with normal mouse immunoglobulins (Ig) of the same immunoglobulin class and concentration as the primary antibody (R&D Systems). The specificity of the immunoreaction was also verified by preabsorption of the antibody with an excess of IL-1RII (20 μg/ml). Thereafter, the strips were incubated for 1 h at 37° C. with Fc-specific peroxidase-labeled goat anti-mouse antibody (1:3000 dilution in the blocking solution; Jackson ImmunoResearch Laboratories, Inc., West Grove, Pa.), washed three times in PBS/0.1% (v/v) Tween 20, incubated for 1 min with an enhanced chemiluminescence system (BM chemiluminescence blotting substrate [POD]; Roche Diagnostics, Laval, PQ, Canada), and exposed to BioMax film (Eastman Kodak, Rochester, N.Y.) for 5-30 sec for an optimal detection(all bands visible but not overexposed).

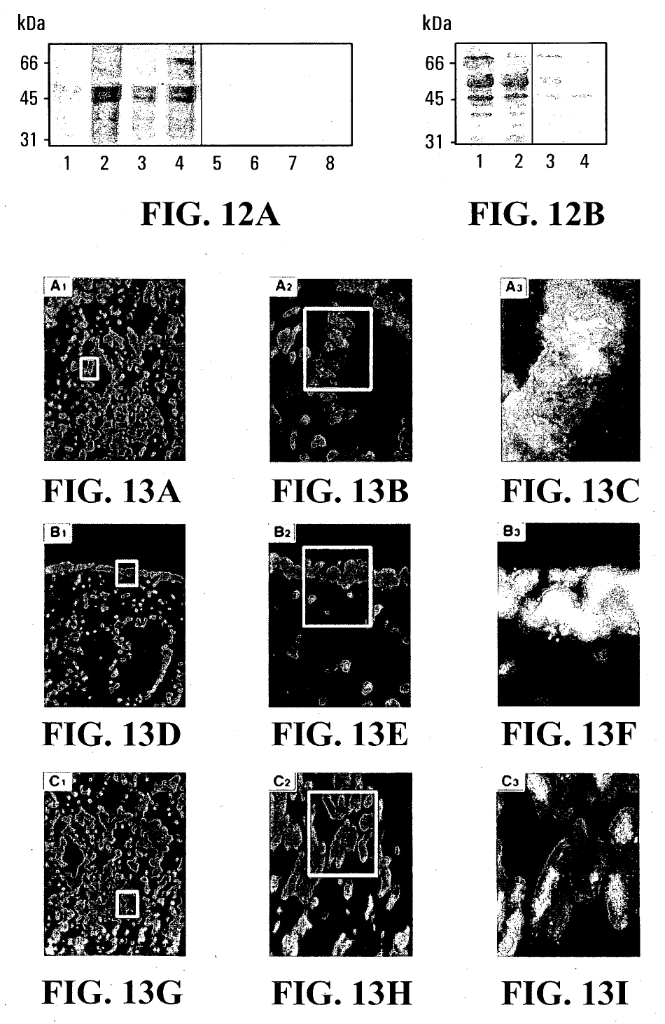

To further examine IL-1RII-protein expression throughout the menstrual cycle, total endometrial proteins were extracted and equivalent amounts were subjected to Western blot analysis. Our results showed that the antibody specifically recognized several major and minor bands (FIG. 12). From these, the 68- and 45-kDa bands are consistent with the commonly reported molecular weights of the membrane bound and the soluble receptors, respectively. The immunoreactive bands were absent when the primary mouse monoclonal anti-IL-1RII antibody was replaced by an equal concentration of normal mouse IgGs (FIG. 12A). Low molecular weight bands (<45 kDa) disappeared when the antibody was preabsorbed with an excess of recombinant IL-1RII (20 μg/ml) before being incubated with nitrocellulose membrane-transferred proteins, whereas the intensity of major higher-molecular-weight bands was considerably reduced (FIG. 12B), suggesting specific interaction with the anti-IL-1RII antibody. As shown in FIG. 12A, all IL-1RII bands revealed by the antibody were markedly intense at the approach of ovulation. The intensity of these bands clearly decreased during the midsecretory phase but increased again thereafter in tissues from late secretory endometria.

### Example 3

### In Situ Hybridization of IL-1RII mRNA in the Endometrium of Healthy Women

Subjects. Women who participated in the study provided informed consent for a protocol approved by the Saint-Francois d'Assise Hospital Ethics Committee on Human Research. These women were aged between 23 and 47 yr (mean±SD, 34.6±5.0 yr). They were fertile, requested tubal ligation, and had a normal and regular menstrual cycle. None had visible endometrial hyperplasia or neoplasia, inflammatory disease, or endometriosis at the time of clinical examination or laparoscopy. Women were not receiving any anti-inflammatory or hormonal medication at least 3 mo before laparoscopy. The cycle day was determined according to the cycle history and histological criteria (Noyes R W, et al, 1995). Eighteen women were in the proliferative phase and 24 in the secretory phase.

Collection of endometrial biopsy specimens. Endometrial biopsy specimens were obtained using sterile pipelle (Unimar, Inc., Neuilly-en-Thelle, France). Samples were placed at 48° C. in sterile Hank balanced salt solution (HBSS; Gibco BRL, Burlington, ON, Canada) containing 100 U/ml of penicillin, 100 mg/ml of streptomycin, and 0.25 mg/ml of amphotericin. Samples were then immediately transported to the laboratory, washed twice in HBSS at 48° C., then snap-frozen on dry ice and kept at −80° C. in Eppendorf tubes for Western blot and reverse transcription-polymerase chain reaction (RT-PCR) analyses or in Tissue-Tek OCT compound (Miles, Inc., Elkhart, Ind.) for immunohistochemical studies.

In situ hybridization was performed as described in our previous studies (Jolicoeur C, et al, 1998). Briefly, cDNA for human IL-1RII, a 1.3-kilobase fragment, was subcloned into the plasmid vector pcDNA3 using the restriction enzymes HindIII and BamHI (Bossu P, et al, 1995). Biotin-labeled cDNA probes were prepared by nick-translation from the entire plasmid vector with the IL-1RII cDNA (Lawrence J B et al, 1985) or from the plasmid vector alone (negative control) using a BioNick Labeling System (Gibco BRL). Serial cryosections were prepared and fixed in formaldehyde as described earlier, then progressively dehydrated in alcohol baths (50%-100% [v/v]). Sections were prehybridized with the hybridization buffer (50% [v/v] formamide, 10% [v/v] dextran sulfate, 0.1% SDS [w/v], 2×SSC [single strength: 0.15 M sodium chloride and 0.015 M sodium citrate], and 1× Denhardt solution [0.02% (v/v) Ficoll (Amersham Pharmacia Biotech, Inc., Baie d'Urfé, QC, Canada), 0.02% (w/v) human serum albumin (HSA), 0.02% (w/v) polyvinylpyrolidone (Sigma), and 40 mM monosodium phosphate (pH 7)]), then hybridized with 5 ng/ml of biotinylated probe in the hybridization buffer. Biotin was detected by a series of 45-min incubations at 37° C. with a rabbit polyclonal antibiotin antibody (1% [v/v] dilution in PBS/0.25% [w/v] HSA; Enzo Diagnostics, Inc., Farmingdale, N.Y.), a biotinylated goat anti-rabbit polyclonal antibody (1% [v/v] dilution in PBS/0.25% [w/v] HSA; Vector Laboratories), and fluorescein isothiocyanate-conjugated streptavidin (0.5% [v/v] in PBS/0.25% [w/v] HSA; Roche Diagnostics, Montreal, PQ, Canada), respectively. Slides were then treated with propidium iodine (1.5 μg/ml of distilled water; Sigma) which makes the nucleus visible in yellow-orange on ultraviolet excitation, and mounted with Mowiol (Calbiochem, San Diego, Calif.), to which p-phenylenediamine (Sigma), an antifading agent, was added at a final concentration of 1 mg/ml. Sections were finally observed under the Leica microscope equipped for fluorescence and connected to an image analysis system (ISIS; Metasystems, Altlussheim, Germany). As negative controls, sections from each tissue were incubated without specific cDNA probes or with nonspecific DNA probes prepared by nick-translation from the plasmid vector alone (i.e., without the IL-1RII cDNA).

Expression of IL-1RII mRNA in the endometrium was studied by in situ hybridization to localize the site of synthesis. FIG. 13 shows the appearance of endometrial glands and stroma at 167× (A1, B1, and C1) and 666× (A2, B2, and C2) magnifications following hybridization and staining with propidium iodine (late proliferative endometrial tissue, Day 13). The hybridization signal (green-yellow) could only be visualized at higher magnification (1665×), as illustrated in the same figure, and was more pronounced in glandular (A3) and surface (B3) epithelial than-in stromal (C3) cells. No hybridization was observed in negative controls including the omission of biotinylated DNA probes prepared from the plasmid containing IL-1RII cDNA insert or the use of biotinylated DNA probes obtained from the plasmid alone.

### Example 4

### Reverse Transcription-Polymerase Chain Reaction of IL-1RII mRNA in Endometrial Biopsies of Healthy Women

Subjects. Women who participated in the study provided informed consent for a protocol approved by the Saint-Francois d'Assise Hospital Ethics Committee on Human Research. These women were aged between 23 and 47 yr (mean±SD, 34.6±5.0 yr). They were fertile, requested tubal ligation, and had a normal and regular menstrual cycle. None had visible endometrial hyperplasia or neoplasia, inflammatory disease, or endometriosis at the time of clinical examination or laparoscopy. Women were not receiving any anti-inflammatory or hormonal medication at least 3 mo before laparoscopy. The cycle day was determined according to the cycle history and histological criteria (Noyes R W, et al, 1995). Eighteen women were in the proliferative phase and 24 in the secretory phase.

Collection of endometrial biopsy specimens. Endometrial biopsy specimens were obtained using sterile pipelle (Unimar, Inc., Neuilly-en-Thelle, France). Samples were placed at 48° C. in sterile Hank balanced salt solution (HBSS; Gibco BRL, Burlington, ON, Canada) containing 100 U/ml of penicillin, 100 mg/ml of streptomycin, and 0.25 mg/ml of amphotericin. Samples were then immediately transported to the laboratory, washed twice in HBSS at 48° C., then snap-frozen on dry ice and kept at −80° C. in Eppendorf tubes for Western blot and reverse transcription-polymerase chain reaction (RT-PCR) analyses or in Tissue-Tek OCT compound (Miles, Inc., Elkhart, Ind.) for immunohistochemical studies.

Reverse Transcription-Polymerase Chain Reaction. Total RNA was extracted from endometrial homogenized tissue with Trizol reagent according to the manufacturer's instructions (Gibco BRL). The cDNA was synthesized using 500 ng of total cellular RNA and 2.5 μM random hexamers in 20 ml of a solution containing 50 mM KCl, 10 mM Tris-HCl, 5 mM MgCl2, 1 mM of each deoxyribonucleotide triphosphate (dNTP), 20 U of RNase inhibitor, and 50 U of reverse transcriptase using Gene Amp PCR Core Kit (Perkin-Elmer, Foster City, Calif.). The reaction was incubated at 25° C. for 15 min, 42° C. for 30 min, and 99° C. for 5 min.

For PCR analysis, 2-μl aliquots of each cDNA were amplified in a final volume of 50 μl containing PCR buffer (10 mmol/L of Tris, 50 mmol/L of KCl, and 1.5 mmol/L of MgCl), 0.2 mmol/L of dNTPs, 2.5 U of Taq DNA Polymerase (New England Biolabs, Beverly, Mass.), and 100 pmol of each IL-1RII primer (forward primer, 5′-TCC ATG TGC AAA TCC TCT CTT-3′ (SEQ ID NO:1); reverse primer, 5′-TCC TGC CGT TCA TCT CAT ACC-3′ (SEQ ID NO:2); amplimer size, 576 base pairs [bp]). To quantify the PCR products comparatively and to confirm the integrity of the RNA, we coamplified a housekeeping gene, glyceraldehyde-phosphate dehydrogenase (GAPDH), in a companion tube (forward primer, 5′-TGA TGA CAT CAA GAA GGT GGT GAA G-3′ (SEQ ID NO:3); reverse primer, 5′-TCC TTG GAG GCC ATG TGG GCC AT-3′ (SEQ ID NO:4); amplimer size, 240 bp). Amplification of IL-1RII was achieved with 30 cycles of 1 min of denaturation at 95° C., 1 min of annealing at 60° C., and 1 min of primer extension at 72° C. Amplification of GAPDH was achieved with 30 cycles of 30 sec of denaturation at 95° C., 30 sec of annealing at 60° C., and 1 min of primer extension at 72° C. These optimal conditions were determined following linearity tests using 1, 2, 5, and 10 μl of the RT reaction volume and 25, 30, and 35 amplification cycles. Amplification of genomic DNA with these primers did not produce a signal, suggesting that the amplification sites crossed at least one intron/exon boundary.

Twenty percent of the PCR volume was then analyzed on a 1% (w/v) agarose gel in the presence of ethidium bromide and transferred to Qiabrane Nylon Plus membranes (Qiagen, Santa Clarita, Calif.). Membranes were dehydrated at 37° C. for 30 min, prehybridized with a hybridization buffer comprised of 5×SSC, 5× Denhardt solution, 50 mM NaH2PO4, 0.5% SDS, 200 μg/ml of salmon sperm DNA, and 50% (v/v) formamide; hybridized in the same buffer, but without Denhardt solution and with ³²P-labeled IL-1RII or GAPDH cDNA; and washed with 1×SSC, 0.2×SSC, and 0.1% (w/v) SDS, respectively, before being exposed to x-ray film (Eastman Kodak) for approximately 1 h.

Specificity of the amplification process was verified by Southern blot hybridization. A negative control (PCR in the absence of cDNA) as well as a positive control (cDNA preparation from human endometrial tissue expressing IL-1RII) were included in each series of IL-1RII or GAPDH amplification.

For each endometrial biopsy, PCR was performed three times. The quantity of the PCR products was determined by densitometric analysis of the intensity of the hybridization signal. The relative level of IL-1RII mRNA normalized to GAPDH mRNA was calculated, and the results were expressed as a percentage of the control value (positive control).

Cell Separation. Endometrial tissue was minced into small pieces and dissociated with coliagenase (Sigma) to separate epithelial glands from fibroblast-like cells as previously reported (Akoum A, et al, 1995). These two cell populations were further purified using Percoll density gradients (Amersham Pharmacia Biotech, Inc.). The purity of epithelial or fibrobiast-like stromal cells was verified morphologically; immunocytochemically on coverslip cultures using antibodies specific to cytokeratins (epithelial cell marker), vimentin (stromal cell marker), smooth muscle α-actin, and factor VIII (endothelial cell marker); and by flow cytometry for the presence of leukocytes using anti-CD45 monoclonal antibodies as previously described (Akoum A, et al, 1995). Cells were kept at −80° C. until use.

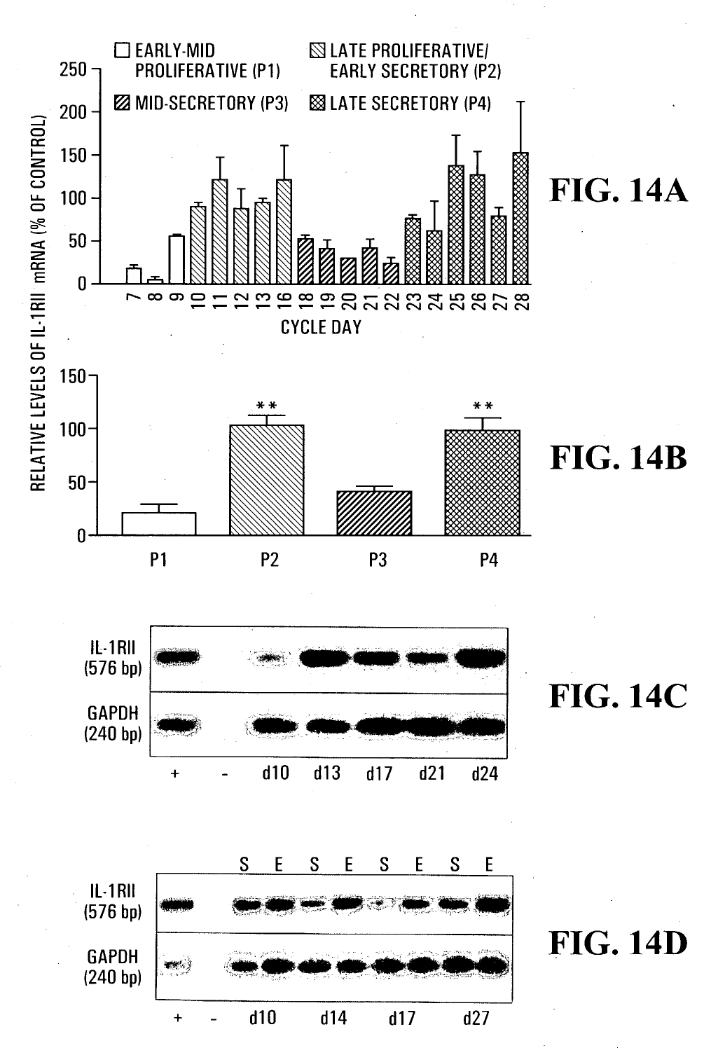

Expression of IL-1RII mRNA throughout the menstrual cycle was studied by semiquantitative RT-PCR. This was achieved by normalizing the IL-1RII mRNA to the mRNA of the coamplified housekeeping gene GAPDH and by including an equal amount of the same preparation of positive control (RT preparation of cDNA from human endometrial RNA) in every series of amplifications. The control, which was subjected to the same experimental conditions from the amplification reaction until Southern blot analysis and autoradiography, allowed for monitoring of the interassay variation. Results were expressed as a percentage of the control value (i.e., the amount of IL-1RII mRNA relative to that of the corresponding GAPDH divided by the amount of IL-1RII mRNA relative to that of GAPDH in the control ×100). Results from 20 endometrial biopsies across the menstrual cycle (FIG. 14A) show that IL-1RII mRNA levels are low in early proliferative endometrial tissues and follow a kinetics of expression comparable to that found for the protein by immunohistochemistry (luminal secretion) and Western blot analysis.

In fact, mRNA levels were elevated in late proliferative, early secretory, and late secretory endometria, but they significantly decreased in tissues from the midsecretory phase. Representative examples of RT-PCR and Southern blot analyses of IL-1RII mRNA in tissues from different cycle phases are shown in FIG. 14B. The RT-PCR analysis of IL-1RII mRNA in separated cells showing higher levels of expression in epithelial than in stromal cells confirmed the in situ hybridization data (FIG. 14C).

### Example 5

### Analysis of MIF Expression in the Endometriotic Lesions

Source and handling of tissue. Endometriotic tissue specimens used in this study were obtained from women who provided informed consent for a research protocol approved by Saint-Francois d'Assise Hospital Ethics Committee on Human Research. These patients were found to have endometriosis during laparoscopy or laparotomy, had no endometrial hyperplasia or neoplasia, and had not received any antiinflammatory or hormonal medication during a period of at least 3 months before surgery. Endometriosis was staged according to the revised American Fertility Society classification system (American Fertility Society, 1985). The cycle phase (proliferative or secretory) was determined according to the patients' cycle history and to the serum progesterone. The mean age was 34.6 plus or minus 6.0 yr. Endometriotic biopsies were immediately placed at 4° C. in sterile HBSS (Life Technologies, Inc., Burlington, Ontario, Canada) containing 100 IU/ml penicillin, 100 μg/ml streptomycin, and 0.25 μg/ml amphotericin transported to the laboratory at which they were immediately washed in HBSS at 4° C., snap frozen on dry ice, or fixed in 10% formalin.

For Western blotting, ELISA, and RT-PCR analyses, 24 biopsies were taken. These biopsies were from women with endometriosis stage I (two typical black-blue, seven red flame-like, and three white), stage II (four typical black-blue, two red flame-like, and three white), and stages III-IV (two red flame-like and one white). Biopsies were all snap frozen and kept at −80° C. in microcentrifuge tubes (Eppendorf, Gordon Technologies Inc., Mississauga, Ontario, Canada) until used.

Western blotting. Protein extraction from endometriotic tissue was performed according to our previously described procedure (Bigonnesse F, et al, 2001), and total protein concentration was determined using the DC protein assay (Bio-Rad Laboratories Ltd., Mississauga, Ontario, Canada). For Western blot analysis, denatured proteins were separated by SDS-PAGE in 15% acrylamide slab gels and transferred onto 0.45-μm nitrocellulose membranes. Equal protein loading was confirmed by staining the membrane with Ponceau S (2%). Nitrocellulose membranes were then cut into strips and incubated overnight at 4 C with a polyclonal goat antihuman MIF antibody (R&D Systems) at 2 μg/ml of blocking solution (0.1 m Tris buffer, 0.9% NaCl/0.05% Tween 20 containing 5% nonfat dry milk [wt/vol]) or with normal goat Ig (R&D Systems) at the same concentration. Strips were then washed in TBS-0.1% Tween 20, incubated for 1 h at room temperature with a peroxidase-conjugated rabbit antigoat antibody (Jackson ImmunoResearch Laboratories, Inc.), diluted 1:10,000 in the blocking solution, washed again in TBS-0.1% Tween 20, incubated for 1 min with an enhanced chemiluminescence system using BM chemiluminescence blotting substrate (Roche Diagnostics), and exposed to BioMax film (Kodak) for several time intervals allowing for an optimal detection (all bands visible but not overexposed).

RT-PCR. Total RNA was extracted from endometriotic tissue with TRIzol reagent (Life Technologies, Inc.) according to the manufacturer's instructions. The GeneAmpPCR core kit (Perkin-Elmer Corp., Foster City, Calif.) was used to synthesize cDNA with 500 ng total cellular RNA and 2.5 μmol/liter random hexamers in 20 μL of a RT-PCR solution (50 mmol/liter KCl, 10 mmol/liter Tris-HCl, 5 mmol/liter MgCl₂, 1 mmol/liter each of dNTPs, 20 IU RNase inhibitor, and 50 IU reverse transcriptase). The reaction was incubated at 25° C. for 15 min, 42° C. for 30 min, and 99° C. for 5 min. For PCR analysis, we used 10% of the reverse transcription (RT) reaction volume as template in a final volume of 50 μL with 50 pmol of each MIF primer (forward primer, 5′-CTCTCCGAGCTCACCCAGCAG-3′ (SEQ ID NO:5); reverse primer, 5′-CGCGTTCATGTCGTAATAGTT-3′ (SEQ ID NO:6); amplimer size, 255 bp), 0.2 mmol/liter dNTP, and 2.5 IU Taq DNA polymerase (QIAGEN, Santa Clarita, Calif.). Amplification was performed for 30 cycles composed of 1 min denaturation (at 94° C.), 1 min annealing (at 60° C.), and 1 min primer extension (at 72° C.). These optimal conditions were determined by performing linearity tests with 5%, 10%, and 20% of the RT reaction volume and 25 and 30 amplification cycles. Amplification of genomic DNA with these primers did not produce a signal, suggesting that the amplification sites crossed at least one intron/exon boundary. Of the PCR volume, 20% was fractionated by electrophoresis in a 1.8% agarose gel in the presence of ethidium bromide and transferred to a Qiabrane Nylon Plus membrane (QIAGEN). Then the membrane was dehydrated at 37° C. for 30 min. prehybridized with a hybridization buffer composed of 5×SSC (0.15 mol/liter sodium chloride and 0.015 mol/liter sodium citrate), 5× Denhardt's solution, 50 mmol/liter NaH2PO4, 0.5% SDS, 200 μg/ml salmon sperm DNA, and 50% formamide; hybridized with ³²P-labeled MIF cDNA in the same buffer except Denhardt's solution; washed with SSC solutions containing 0.1% SDS, 11×SSC, 0.2×SSC and 0.1×SSC, respectively, and exposed to x-ray film (Eastman Kodak Co.) for different time intervals allowing for an optimal detection (signals visible but not overexposed). As control, glyceraldehyde phosphate dehydrogenase (GAPDH) amplification was used. For PCR analysis, we used 25% of the RT reaction volume as template in a final volume of 50 μl with 25 μmol of each primer (forward primer, 5′-TGATGACATCAAGAAGGTGGTGAAG-3′ (SEQ ID NO:3); reverse primer, 5′-TCCTTGGAGGCCATGTGGGCCAT-3′ (SEQ ID NO:4); amplimer size, 240 bp), 0.2 mmol/liter dNTPs, and 1 IU Vent DNA polymerase. Amplification was performed for 30 cycles of 30 sec denaturation (at 95° C.), 30 sec annealing (at 60° C.), and 1 min primer extension (at 72° C.). These optimal conditions were determined following linearity tests using 10%, 25%, 50%, and 75% of the RT reaction volume. Specificity of the amplification process was verified by Southern blot hybridization. A negative control (PCR in the absence of cDNA) as well as a positive control (cDNA preparation from the human hystiocytic cell line U937, known to secrete MIF) were included in each series of MIF or GAPDH amplification. The intensity of the hybridization signals was determined by computer-assisted densitometry, using Quantity One Quantitation software (Bio-Rad Laboratories, Inc.). The quantity of the PCR products was determined by densitometric analysis of the intensity of the hybridization signal. The relative level of MIF mRNA normalized to GAPDH mRNA was calculated, and the results were expressed as percent of control (positive control).

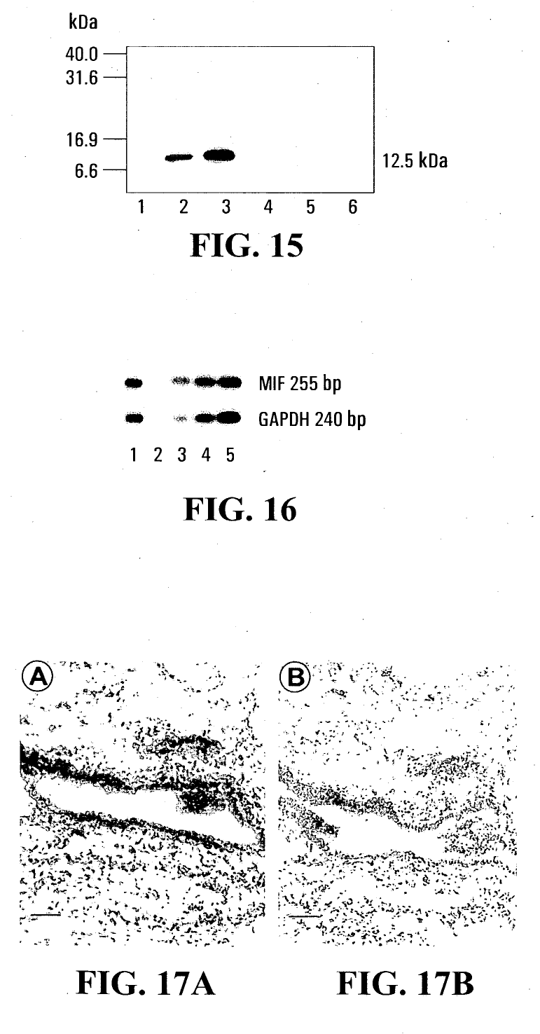

One objective of this study was to assess the presence of MIF in endometriotic lesions. Western blot analysis of proteins extracted from endometriotic tissue using a goat polyclonal antihuman-MIF antibody showed a single specific 12.5-kDa band corresponding to the known molecular weight of MIF (FIG. 15). RT-PCR and Southern blot analysis showed specific MIF transcripts, thereby confirming MIF expression in endometriotic tissue at the level of mRNA (FIG. 16).

### Example 6

### Immunohistochemical Analysis of MIF in Endometriotic Lesions

Source and handling of tissue. Endometriotic tissue specimens used in this study were obtained from women who provided informed consent for a research protocol approved by Saint-Francois d'Assise Hospital Ethics Committee on Human Research. These patients were found to have endometriosis during laparoscopy or laparotomy, had no endometrial hyperplasia or neoplasia, and had not received any antiinflammatory or hormonal medication during a period of at least 3 months before surgery. Endometriosis was staged according to the revised American Fertility Society classification system (American Fertility Society, 1985). The cycle phase (proliferative or secretory) was determined according to the patients' cycle history and to the serum progesterone. The mean age was 34.6 plus or minus 6.0 yr. Endometriotic biopsies were immediately placed at 4° C. in sterile HBSS (Life Technologies, Inc., Burlington, Ontario, Canada) containing 100 IU/ml penicillin, 100 μg/ml streptomycin, and 0.25 μg/ml amphotericin transported to the laboratory at which they were immediately washed in HBSS at 4° C., snap frozen on dry ice, or fixed in 10% formalin.

For immunohistochemical analysis, 25 endometriotic biopsies were included. Fifteen were frozen at −80° C. in Tissue-Tek OCT compound (Miles Inc., Elkhart, Ind.), and 10 were embedded in paraffin. These biopsies were from women with endometriosis stage I (two typical black-blue, two red flame-like), stage II (two typical black-blue, three white, and one from the inner wall of ovarian endometrioma), stages III-IV (three typical black-blue, three white, and nine from the inner wall of ovarian endometrioma).

Immunohistochemistry. Five-micrometer cryosections of Optimal Cutting Temperature-frozen endometriotic lesions were mounted on poly-1-lysine-coated microscope glass slides, fixed during 20 min in a 10% buffered formalin phosphate solution (Fisher Scientific, Montreal, Quebec, Canada), and washed in PBS. Five-micrometer sections of paraffin-embedded tissues were mounted on poly-1-lysine-coated microscope glass slides, deparaffinized in toluene, rehydrated through graded solutions of ethanol and water, and washed in PBS. The subsequent steps were the same for cryosections and paraffin-embedded tissue sections. Briefly, after permeabilization with Triton-X-100 (1% in PBS) and elimination of endogenous peroxidase with H₂O₂ (0.3% in absolute methanol), tissue sections were successively incubated at room temperature for 90 min with a goat polyclonal antihuman-MIF antibody (R&D Systems, Minneapolis, Minn.) [0.66 μg/ml in PBS/0.2% BSA/0.01% Tween 20 (PBS/BSA/Tween)], 90 min with a biotin-conjugated rabbit antigoat antibody (Jackson ImmunoResearch Laboratories, Inc., West Grove, Pa.) (1:500 in PBS/BSA/Tween, 45 min with peroxidase-conjugated streptavidin (Jackson ImmunoResearch Laboratories, Inc.) (1:500 dilution in PBS/BSA/Tween) and 20 min diaminobenzidine used as chromogen (3 mg diaminobenzidine/0.03% H₂O₂ in PBS). Sections were counterstained with hematoxylin and mounted in Mowiol (Calbiochem-Novabiochem Corp., La Jolla, Calif.). Sections incubated with goat IgGs at the same concentration as the primary antibody were used as negative controls in all experiments. Slides were viewed using a microscope (mikroskopie und systeme GmbH, model DMRB, Leica Corp., Postfach, Wetzlar, Germany) and photographed with 100 ASA film (Eastman Kodak Co., Rochester, N.Y.).

Immunohistochemical analysis of MIF expression showed a specific brownish immunostaining localized to specific compartments of endometriotic tissue. MIF was found to be strongly expressed in glandular epithelial cells and in cells scattered throughout the stroma (FIG. 17A). Incubation of tissue sections with normal goat IgGs used at concentration equivalent to that of the primary goat polyclonal anti-MIF antibody (negative control) did not result in any nonspecific immunostaining (FIG. 17B).

### Example 7

### Analysis of the MIF Protein Expression in Specific Endometriotic Cells

Source and handling of tissue. Endometriotic tissue specimens used in this study were obtained from women who provided informed consent for a research protocol approved by Saint-Francois d'Assise Hospital Ethics Committee on Human Research. These patients were found to have endometriosis during laparoscopy- or laparotomy, had no endometrial hyperplasia or neoplasia, and had not received any antiinflammatory or hormonal medication during a period of at least 3 months before surgery. Endometriosis was staged according to the revised American Fertility Society classification system (American Fertility Society, 1985). The cycle phase (proliferative or secretory) was determined according to the patients' cycle history and to the serum progesterone. The mean age was 34.6 plus or minus 6.0 yr. Endometriotic biopsies were immediately placed at 4° C. in sterile HBSS (Life Technologies, Inc., Burlington, Ontario, Canada) containing 100 IU/ml penicillin, 100 μg/ml streptomycin, and 0.25 μg/ml amphotericin transported to the laboratory at which they were immediately washed in HBSS at 4° C., snap frozen on dry ice, or fixed in 10% formalin.

Dual immunofluorescent staining. Cryostat and paraffin-embedded tissue sections were treated and incubated at room temperature for 120 min with a goat polyclonal antihuman-MIF antibody (R&D Systems, Minneapolis, Minn.) at 0.66 μg/ml in PBS/BSA/Tween as described earlier (see the Immunohistochemistry methodology). After a PBS/0.05% Tween 20 rinse, sections were incubated at room temperature for 90 min with one of the following antibodies: mouse monoclonal antihuman-CD68 (DAKO Corp. Diagnostics Canada Inc., Mississauga, Ontario, Canada) (diluted 1:50 in PBS/BSA/Tween) to detect macrophages; mouse monoclonal antihuman-CD3 (diluted 1:100 in PBS/BSA/Tween) to detect T lymphocytes; and rabbit polyclonal antihuman von Willebrandt factor (vWF) (Sigma-Aldrich Corp. Canada LTD, Oakville, Ontario, Canada) (diluted 1:200 in PBS/BSA/Tween) to detect endothelial cells. After a subsequent wash in PBS/0.05% Tween 20, tissue sections were incubated simultaneously for 60 min at room temperature in the dark with fluorescein isothiocyanate-conjugated donkey antigoat antibody (Jackson ImmunoResearch Laboratories, Inc.) (diluted 1:50 in PBS/BSA/Tween) and rhodamine-conjugated sheep antimouse antibody (Roche Diagnostics, Laval, Quebec, Canada) (diluted 1:10 in PBS/BSA/Tween) for tissues marked for MIF and CD68 or CD3 or rhodamine-conjugated mouse antirabbit antibody (Jackson ImmunoResearch Laboratories, Inc.) (diluted 1:50 in PBS/BSA/Tween) for tissues marked for MIF and vWF. After a final wash in PBS/0.05% Tween 20, slides were mounted in Mowiol containing 10% para-phenylenediamine (Sigma-Aldrich Corp. Canada Ltd.), an antifading agent, and observed under the microscope (Leica Corp.) equipped for fluorescence with a 100-W UV lamp. Photomicrographs were taken with 400 ASA film (Kodak).

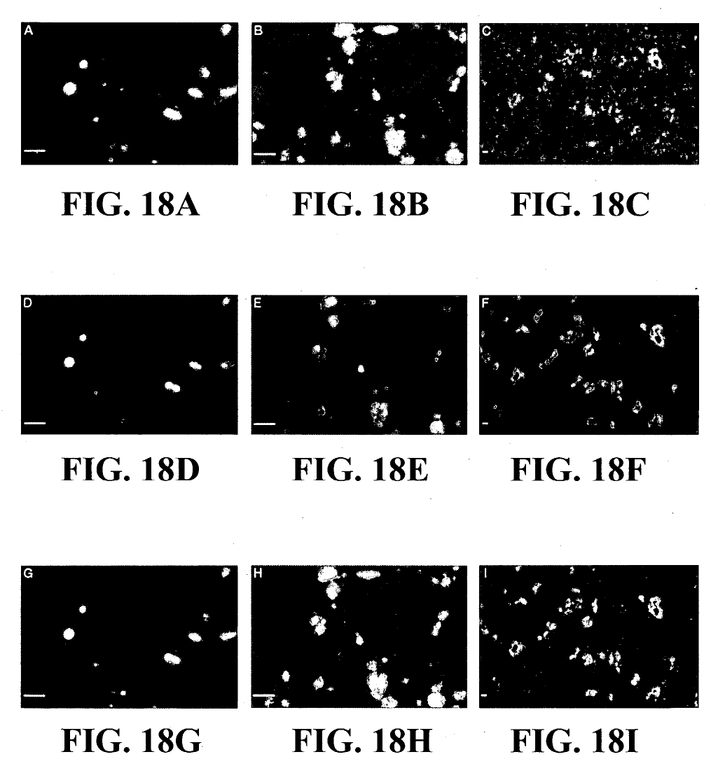

To identify cells expressing MIF in the stroma, dual immunofluorescence analysis was performed using antibodies specific to MIF and to CD3, CD68, and vWF. Representative photomicrographs exhibited in FIG. 18 show a marked expression of MIF in CD3-positive T lymphocytes, CD68-positive macrophages, and vWF-positive endothelial cells.

### Example 8

### Analysis of MIF Expression in Different Endometriotic Lesions and Disease Stage

Source and handling of tissue. Endometriotic tissue specimens used in this study were obtained from women who provided informed consent for a research protocol approved by Saint-Francois d'Assise Hospital Ethics Committee on Human Research. These patients were found to have endometriosis during laparoscopy or laparotomy, had no endometrial hyperplasia or neoplasia, and had not received any antiinflammatory or hormonal medication during a period of at least 3 months before surgery. Endometriosis was staged according to the revised American Fertility Society classification system (American Fertility Society, 1985). The cycle phase (proliferative or secretory) was determined according to the patients' cycle history and to the serum progesterone. The mean age was 34.6 plus or minus 6.0-yr. Endometriotic biopsies were immediately placed at 4° C. in sterile HBSS (Life Technologies, Inc., Burlington, Ontario, Canada) containing 100 IU/ml penicillin, 100 μg/ml streptomycin, and 0.25 μg/ml amphotericin transported to the laboratory at which they were immediately washed in HBSS at 4° C., snap frozen on dry ice, or fixed in 10% formalin.

For Western blotting, ELISA, and RT-PCR analyses, 24 biopsies were taken. These biopsies were from women with endometriosis stage I (two typical black-blue, seven red flame-like, and three white), stage II (four typical black-blue, two red flame-like, and three white), and stages III-IV (two red flame-like and one white). Biopsies were all snap frozen and kept at −80° C. in microcentrifuge tubes (Eppendorf, Gordon Technologies Inc., Mississauga, Ontario, Canada) until used.

MIF ELISA. MIF concentration in endometriotic tissue protein extract was measured by ELISA according to a previously reported procedure (Calandra T, et al, 1995). Briefly, this technique uses a capture mouse monoclonal antihuman-MIF antibody (R&D Systems), a rabbit polyclonal antihumah-MIF antibody for detection, alkaline phosphatase-conjugated goat antirabbit IgGs (Chemicon International Inc., Temecula, Calif.) and paranitrophenyl phosphate as substrate (Sigma). The optical density was measured at 405 nm and MIF concentrations were extrapolated from a standard curve using recombinant human MIF.

RT-PCR. Total RNA was extracted from endometriotic tissue with TRIzol reagent (Life Technologies, Inc.) according to the manufacturer's instructions. The GeneAmpPCR core kit (Perkin-Elmer Corp., Foster City, Calif.) was used to synthesize cDNA with 500 ng total cellular RNA and 2.5 μmol/liter random hexamers in 20 μL of a RT-PCR solution (50 mmol/liter KCl, 10 mmol/liter Tris-HCl, 5 mmol/liter MgCl2, 1 mmol/liter each of dNTPs, 20 IU RNase inhibitor, and 50 IU reverse transcriptase). The reaction was incubated at 25° C. for 15 min, 42° C. for 30 min, and 99° C. for 5 min. For PCR analysis, we used 10% of the reverse transcription (RT) reaction volume as template in a final volume of 50 μL with 50 pmol of each MIF primer (forward primer, 5′-CTCTCCGAGCTCACCCAGCAG-3′ (SEQ ID NO:5); reverse primer, 5′-CGCGTTCATGTCGTAATAGTT-3′ (SEQ ID NO:6); amplimer size, 255 bp), 0.2 mmol/liter dNTP, and 2.5 IU Taq DNA polymerase (QIAGEN, Santa Clarita, Calif.). Amplification was performed for 30 cycles composed of 1 min denaturation (at 94° C.), 1 min annealing (at 60° C.), and 1 min primer extension (at 72° C.). These optimal conditions were determined by performing linearity tests with 5%, 10%, and 20% of the RT reaction volume and 25 and 30 amplification cycles. Amplification of genomic DNA with these primers did not produce a signal, suggesting that the amplification sites crossed at least one intron/exon boundary. Of the PCR volume, 20% was fractionated by electrophoresis in a 1.8% agarose gel in the presence of ethidium bromide and transferred to a Qiabrane Nylon Plus membrane (QIAGEN). Then the membrane was dehydrated at 37° C. for 30 min, prehybridized with a hybridization buffer composed of 5×SSC (0.15 mol/liter sodium chloride and 0.015 mol/liter sodium citrate), 5× Denhardt's solution, 50 mmol/liter NaH₂PO₄, 0.5% SDS, 200 μg/ml salmon sperm DNA, and 50% formamide; hybridized with 32P-labeled MIF cDNA in the same buffer except Denhardt's solution; washed with SSC solutions containing 0.1% SDS, 11×SSC, 0.2×SSC and 0.1×SSC, respectively, and exposed to x-ray film (Eastman Kodak Co.) for different time intervals allowing for an optimal detection (signals visible but not overexposed). As control, glyceraldehyde phosphate dehydrogenase (GAPDH) amplification was used. For PCR analysis, we used 25% of the RT reaction volume as template in a final volume of 50 μl with 25 pmol of each primer (forward primer, 5′-TGATGACATCAAGAAGGTGGTGAAG-3′ (SEQ ID NO:3); reverse primer, 5′-TCCTTGGAGGCCATGTGGGCCAT-3′ (SEQ ID NO:4); amplimer size, 240 bp), 0.2 mmol/liter dNTPs, and 1 IU Vent DNA polymerase. Amplification was performed for 30 cycles of 30 sec denaturation (at 95° C.), 30 sec annealing (at 60° C.), and 1 min primer extension (at 72° C.). These optimal conditions were determined following linearity tests using 10%, 25%, 50%, and 75% of the RT reaction volume. Specificity of the amplification process was verified by Southern blot hybridization. A negative control (PCR in the absence of cDNA) as well as a positive control (cDNA preparation from the human hystiocytic cell line U937, known to secrete MIF) were included in each series of MIF or GAPDH amplification. The intensity of the hybridization signals was determined by computer-assisted densitometry, using Quantity One Quantitation software (Bio-Rad Laboratories, Inc.). The quantity of the PCR products was determined by densitometric analysis of the intensity of the hybridization signal. The relative level of MIF mRNA normalized to GAPDH mRNA was calculated, and the results were expressed as percent of control (positive control).

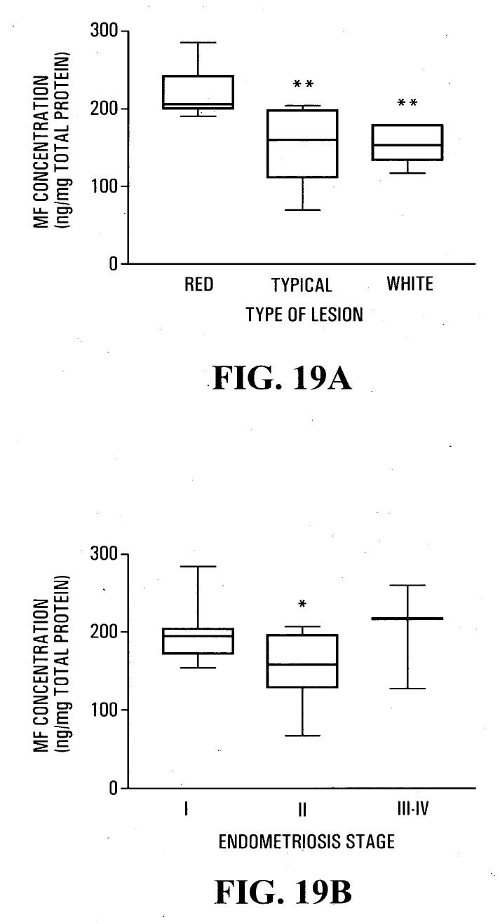

To quantify MIF expression in endometriotic tissue and examine whether MIF expression correlates with the type of endometriotic lesion and endometriosis stage, we measured MIF concentrations in total protein extracts by ELISA and determined the levels of mRNA in the same tissues using a semiquantitative RT-PCR analysis. As shown in FIG. 19A, the highest concentrations of MIF protein were found in flamelike red endometriotic lesions, compared with typical black bluish (P<0.01) or with white lesions (P<0.01). Otherwise, no significant difference between typical and white endometriotic lesions was found. Furthermore, MIF concentrations appeared to be significantly higher in lesions from endometriosis stage I, compared with those from endometriosis stage II (P<0.05), whereas no significant difference between stages I and III-IV or II and III-IV was noted. However, only three biopsies from endometriosis stages III-IV were included in this assay, which may have limited the power of statistical analyses including these stages (FIG. 19B). Analysis of MIF protein expression according to the phase of the menstrual cycle showed no statistically significant difference between lesions from the proliferative and the secretory phases.

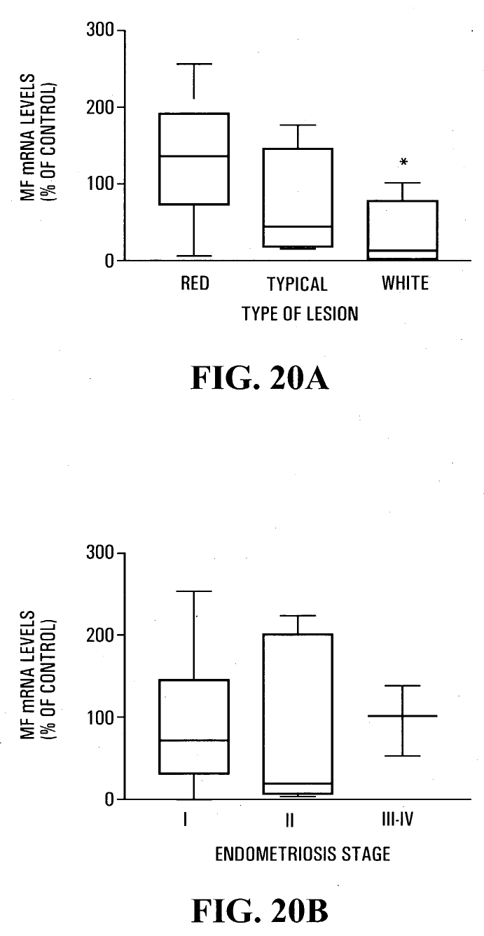

Semiquantitative RT-PCR analysis of MIF mRNA levels in the same endometriotic tissues showed a pattern of expression comparable with that of the protein, but the difference in MIF mRNA levels was found to be significant only between red and white endometriotic lesions (FIG. 20A). On the other hand, no significant difference in MIF mRNA levels according to endometriosis stage (20B) or to the menstrual cycle phase was noted.

### Example 9

### In situ Hybridizatoin of IL-1RII mRNA of Endometrium Biopsies from Healthy Woman and Woman Affected with Endometriosis

Subjects and Tissue Collection. Endometrial tissues were obtained from 79 women aged between 30 and 36 yr who were undergoing laparoscopic surgery for infertility, pelvic pain, or tubal ligation and who had not received any anti-inflammatory or hormonal medication during a period of at least 3 mo before laparoscopy. Fifty-three women had endometriosis of various stages (I, II, III, and IV) according to the revised American Fertility Society classification (Rock J A, 1995). Twenty-six women were fertile and had no visible endometriosis at laparoscopy (Table 9). The cycle phase was determined according to the cycle history, progesterone levels in the serum, and histologic criteria. An informed consent was obtained from each patient, and the study was-approved by the ethical committee of the “Centre Hospitalier Universitaire de Quebec” (CHUQ).

TABLE 9
Clinical characteristics of patients at laparoscopy.

| | | | Subjects by cycle | |
|---|---|---|---|---|
| | Subjects | | phase (n) | |
| | (n) | Age (yr)^{a} | Proliferative | Secretory |
| Controls | 26 | 35.2 ± 6.1 | 9 | 17 |
| Endometriosis (total) | 53 | 31.6 ± 5.8 | 24 | 29 |
| Stage I | 22 | 31.5 ± 6.8 | 11 | 11 |
| Stage II | 19 | 30.5 ± 5.0 | 8 | 11 |
| Stages III-V | 12 | 33.5 ± 4.7 | 5 | 7 |
| Fertile | 24 | 32.5 ± 6.9 | 11 | 13 |
| Infertile | 29 | 30.9 ± 4.7 | 13 | 16 |

^{a}Mean ± SD.

Endometrial samples were collected with a curette before laparoscopy. The tissue was placed in cold, sterile Hanks buffered saline containing antibiotics, then immediately transported to the laboratory and snap-frozen in liquid nitrogen before being stored at −80° C.

Fluorescence In Situ Hybridization. The present experiments were performed as previously described (Jolicoeur C, et al, 1998). Briefly, biotin-labeled IL-1RII cDNA probe was prepared by nick translation (Lawrence J B, et al, 1985) from the entire plasmid vector pcDNA3.

Cryosections (thickness, 5 μm) from 79 endometrial tissues (Table 9)were fixed with formaldehyde and dehydrated with alcohol before being hybridized with 5 ng/ml of biotinylated IL-1RII cDNA probe. Biotin was then detected using a rabbit antibiotin antibody, a biotinylated goat antirabbit antibody, and fluorescein isothiocyanate-conjugated streptavidin, respectively. Sections were finally treated with propidium iodine, which makes the nucleus visible in yellow-orange following ultraviolet excitation, and were observed under a fluorescence microscope (Leica mikroskbpie und systeme GmbH, Model DMRB, Postfach, Wetzlar, Germany). Serial sections from each tissue incubated without IL-1RII cDNA probe or with nonspecific DNA probes prepared from the plasmid vector alone were used as negative controls.

Evaluation of Staining. Staining was evaluated using an arbitrary scale as previously reported (Jolicoeur C, et al, 1998). In brief, each endometrial section was graded according to the intensity of staining three times in three different, randomly selected areas and scored from 0 to 3 (0=absent, 1=light, 2=moderate, and 3=intense). Grading of each specimen was performed by two different observers who had no knowledge of the clinical status of the patients including laparoscopic diagnosis.

Statistical Analyses. The intensity of IL-1RII mRNA hybridization signals was expressed as arbitrary units. Statistical analysis was performed by the Fisher exact probability test (Guzick D S, 1996), and Bonferroni correction was applied when more than two groups were compared. Analysis of IL-RII mRNA levels as determined by semiquantitative RT-PCR was performed using one-way ANOVA and the Tukey test for post-hoc multiple comparisons. All analyses were carried out using the Statistical Analysis System (SAS Institute, Inc., Cary, N.C.). Differences were considered to be statistically significant at P<0.05.

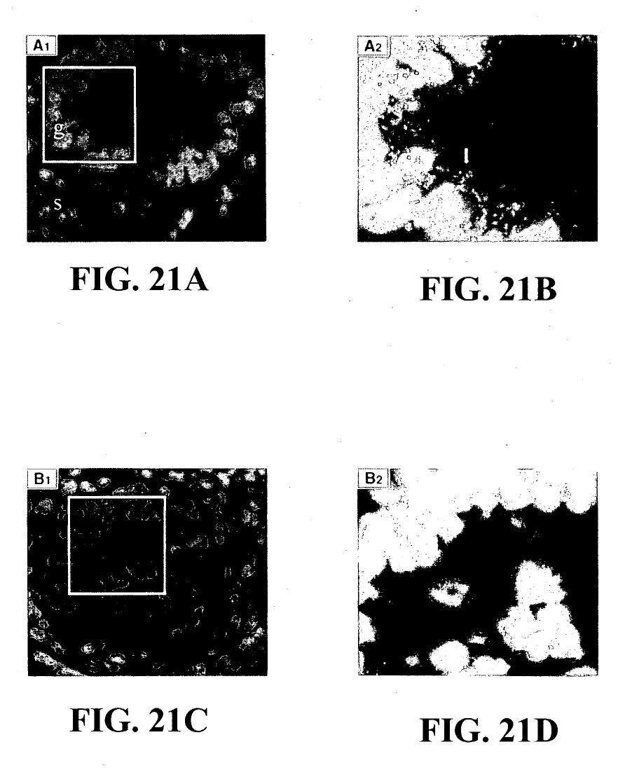

Analysis of IL-1RII Gene Expression in the Endometrium by In Situ Hybridization. The expression of IL-1RII mRNA in the endometrium was studied by in situ hybridization to examine the site of IL-1RII synthesis and to compare the levels of IL-1RII mRNA in patients with and without endometriosis. FIG. 21 shows the appearance of endometrial stroma and glands at 666× magnification (A1 and B1) following hybridization and staining with propidium iodine. The hybridization signal (green-yellow) could only be visualized at higher magnification (1665×) and appeared to be located mainly in the endometrial glands (A2 and B2).

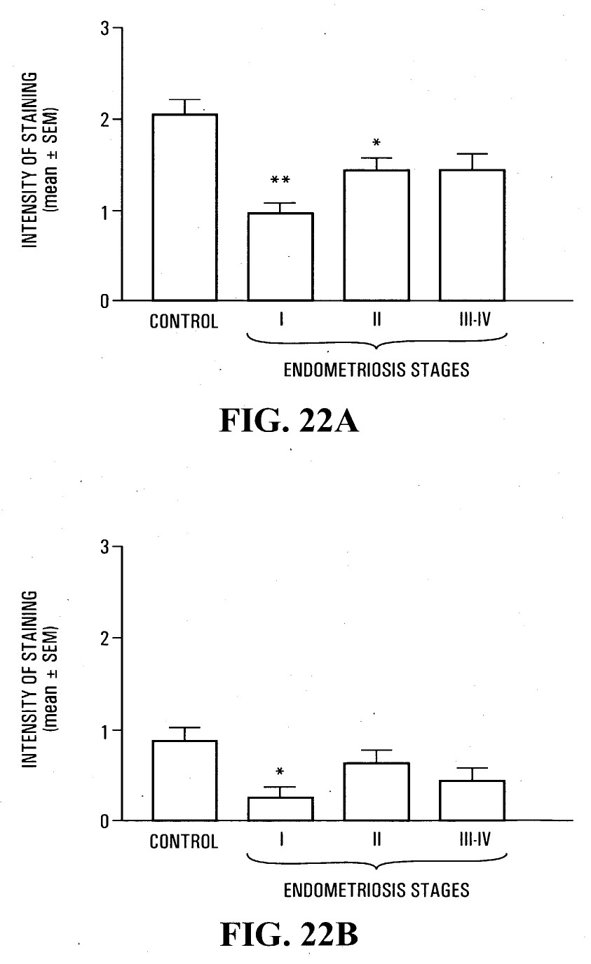

As described earlier, an arbitrary score was used to quantify the IL-1RII mRNA hybridization signal. Statistical analysis of hybridization scores using the Fisher exact test showed a significant decrease in women with endometriosis compared to normal women, both in endometrial glands (P<0.0001) and stroma (P<0.006) (Table 10). Furthermore, when patients with endometriosis were grouped according to the stage of disease, a significant decrease in IL-1RII mRNA expression in the glandular (P<0.0003) as well as the stromal (P<0.012) compartment was observed in stage I. In stage II, a significant decrease in IL-1RII mRNA levels was also observed, but only in the glands (P<0.042), whereas in more advanced stages (III and IV), no statistically significant difference was found. A graphical illustration of IL-1RII mRNA scores in normal controls and in women at different stages of endometriosis is shown in FIG. 22.

The effect of the menstrual cycle on levels of IL-1RII mRNA in the endometrium was also evaluated. Statistical analysis of the hybridization scores showed no significant difference between the proliferative and the secretory phases within the control or the endometriosis groups. However, the decreased expression of IL-1RII mRNA observed in women with endometriosis was more noticeable during the secretory phase of the menstrual cycle, either in the glands (P<0.003) or in the stroma, in which a statistically significant difference between women with and women without endometriosis was seen only during the secretory phase (P<0.018) (Table 10).

TABLE 10
Number of subjects according to intensity of the
IL-1RII mRNA hybridization signal in the endometrium.

| | | Stroma (n) | | | | | Glands (n) | | | | |
|---|---|---|---|---|---|---|---|---|---|---|---|
| | | Intensity of | | | | | Intensity of | | | | |
| | | staining | | | | | staining | | | | |
| | Number | 0 | 1 | 2 | 3 | P^{a} | 0 | 1 | 2 | 3 | P^{a} |
| Controls | 26 | 8 | 13 | 5 | 0 | | 1 | 6 | 10 | 9 | |
| Endometriosis (total) | 53 | 31 | 21 | 1 | 0 | 0.006^{b} | 4 | 35 | 12 | 2 | 0.0001^{b} |
| Stage I^{b} | 22 | 16 | 6 | 0 | 0 | 0.012^{b} | 4 | 15 | 3 | 0 | 0.0003^{b} |
| Stage II^{b} | 19 | 8 | 10 | 1 | 0 | 0.422 | 0 | 12 | 6 | 1 | 0.042^{b} |
| Stages III-IV^{b} | 12 | 7 | 5 | 0 | 0 | 0.5 | 0 | 8 | 3 | 1 | 0.186 |
| Fertile | 23 | 14 | 9 | 0 | 0 | 0.018^{b} | 2 | 13 | 7 | 1 | 0.030^{b} |
| Infertile | 30 | 17 | 12 | 1 | 0 | 0.16 | 2 | 22 | 5 | 1 | 0.0004^{b} |
| Proliferative phase | | | | | | | | | | | |
| Control | 9 | 2 | 6 | 1 | 0 | | 0 | 3 | 2 | 4 | |
| Endometriosis | 24 | 13 | 10 | 1 | 0 | 0.217 | 2 | 15 | 7 | 0 | 0.009^{b} |
| Secretory Phase | | | | | | | | | | | |
| Control | 17 | 6 | 7 | 4 | 0 | | 1 | 3 | 8 | 5 | |
| Endometriosis | 29 | 18 | 11 | 0 | 0 | 0.018^{b} | 2 | 20 | 5 | 2 | 0.003^{b} |

^{a}Fisher exact test.
^{b}Comparison with controls; P values corrected by the Bonferroni procedure.

Statistical analysis of the hybridization scores according to the fertility status of subjects showed that, compared to normal fertile women, fertile women with endometriosis had decreased expression of IL-1RII mRNA, both in the glandular (P<0.030) and in the stromal (P<0.018) compartments of endometrial tissue. However, in infertile women with endometriosis, a significant decrease in IL-1RII mRNA expression was observed only in the glands (P<0.0004) (Table 10).

### Example 10

### RT-PCR Analysis of IL-1RII mRNA Expression in the Endometrium of Healthy Women and Women Suffering from Endometriosis

Subjects and Tissue Collection. Endometrial tissues were obtained from 79 women aged between 30 and 36 yr who were undergoing laparoscopic surgery for infertility, pelvic pain, or tubal ligation and who had not received any anti-inflammatory or hormonal medication during a period of at least 3 mo before laparoscopy. Fifty-three women had endometriosis of various stages (I, II, III, and IV) according to the revised American Fertility Society classification (Rock J A, 1995). Twenty-six women were fertile and had no visible endometriosis at laparoscopy (Table 9). The cycle phase was determined according to the cycle history, progesterone levels in the serum, and histologic criteria. An informed consent was obtained from each patient, and the study was approved by the ethical committee of the “Centre Hospitalier Universitaire de Quebec” (CHUQ).

Endometrial samples were collected with a curette before laparoscopy. The tissue was placed in cold, sterile Hanks buffered saline containing antibiotics, then immediately transported to the laboratory and snap-frozen in liquid nitrogen before being stored at −80° C. Reverse Transcription-Polymerase Chain Reaction. Total RNA from the endometrial tissue of 8 normal women (3 in the proliferative phase and 5 in the secretory phase) and of 10 women with stage I-II endometriosis (4 in the proliferative phase and 6 in the secretory phase) was extracted using a Trizol reagent according to the manufacturer's instructions (Gibco BRL, Burlington, ON, Canada). Total RNA (500 ng) was reverse transcribed into cDNA using 50 U of reverse transcriptase in the presence of random hexamer primers (2.5 mM), dNTPs (1 mM each), 1 U/ml of RNase inhibitor, 10 mM Tris-HCl, 50 mM KCl, and 5 mM MgCl₂ (Gene Amp PCR Core Kit; Perkin-Elmer, Foster City, Calif.). The reaction was incubated at 25° C. for 15 min, 42° C. for 30 min, and 99° C. for 5 min. Two microliters of the reverse transcription (RT) reaction were used for polymerase chain reaction (PCR) in a final volume of 50 μl with 100 pmol of each IL-1RII primer (5′-TCC ATG TGC AAA TCC TCTCTT-3′ (SEQ ID NO:1); 5′-TCC TGC CGT TCA TCT CAT ACC-3′ (SEQ ID NO:2); expected amplimer length, 576 bp), 0.2 mM dNTPs, 2 mM MgCl2, and 2.5 U of Taq polymerase (Groves R W, et al, 1994). Amplification was performed for 30 cycles consisting of 1 min of denaturation (94° C.), 30 sec of annealing (60° C.), and 1 min of primer extension (72° C.). Glyceraldehyde phosphate dehydrogenase (GAPDH) was used as a control. Four microliters of the RT reaction were used for PCR in a final volume of 50 ml with 25 pmol of each primer (5′-TGA TGA CAT CAA GAA GGT GGT GAA G-3′ (SEQ ID NO:3); 5′-TCC TTG GAG GCC ATG TGG GCC AT-3′ (SEQ ID NO:4); amplimer size 240 bp), 0.2 mM dNTPs, and 1 U of Vent DNA Polymerase (New England Biolabs, Beverly, Mass.). Amplification was performed for 30 cycles consisting of 30 sec of denaturation (95° C.), 30 sec of annealing (60° C.), and 1 min of primer extension (72° C.). These optimal conditions were determined following linearity tests using 1, 2, 4, and 8 μl of the RT reaction volume and 25, 30, and 35 amplification cycles. Amplification of genomic DNA with these primers did not produce a signal, suggesting that the amplification sites crossed at least one intron/exon boundary. A total of 20% of the PCR volume was then analyzed on a 1% (w/v) agarose gel in the presence of ethidium bromide and transferred to Qiabrane Nylon Plus membranes (Qiagen, Santa Clarita, Calif.). Membranes were dehydrated at 37° C. for 30 min, prehybridized with a hybridization buffer, hybridized in the same buffer (without Denhardt solution) with ³²P-labeled IL-1RII or GAPDH cDNA, and washed in 1×0.15 M sodium chloride and 0.015 M sodium citrate (SSC), 0.2×SSC, and 0.1% SDS, respectively, before being exposed to x-ray film (Eastman Kodak, Rochester, N.Y.).

Specificity of the amplification process was verified by Southern blot hybridization. A negative control (PCR in the absence of cDNA) as well as a positive control (cDNA preparation from human endometrial tissue expressing IL-1RII) were included in each series of IL-1RII or GAPDH amplification. The quantity of the PCR products was determined by densitometric analysis of the intensity of the hybridization signal. The relative level of IL-1RII mRNA normalized to GAPDH mRNA was calculated, and the results were expressed as a % of the control value (positive control).

Statistical Analyses. The intensity of IL-1RII mRNA hybridization signals was expressed as arbitrary units. Statistical analysis was performed by the Fisher exact probability test (Guzick D S, 1996), and Bonferroni correction was applied when more than two groups were compared. Analysis of IL-RII mRNA levels as determined by semiquantitative RT-PCR was performed using one-way ANOVA and the Tukey test for post-hoc multiple comparisons. All analyses were carried out using the Statistical Analysis System (SAS Institute, Inc., Cary, N.C.). Differences were considered to be statistically significant at P,<0.05.

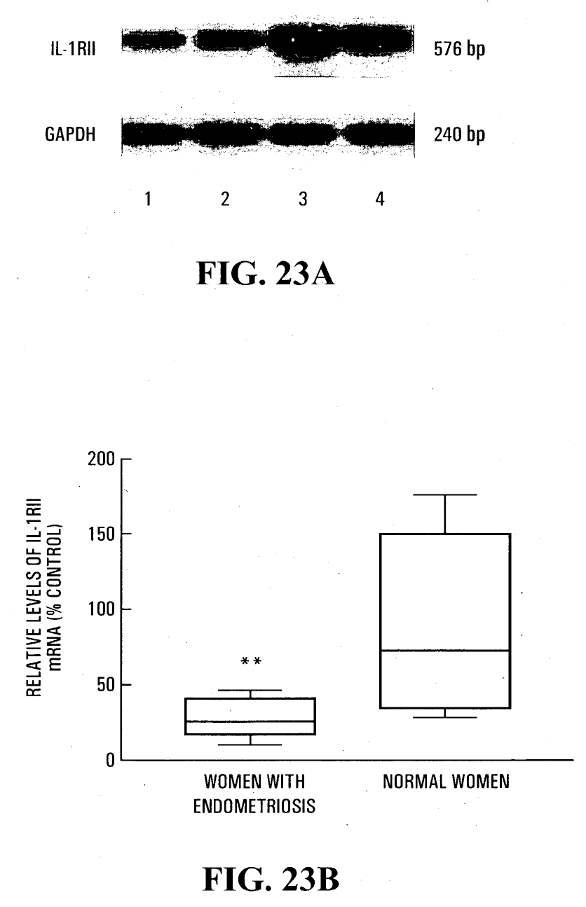

Expression of IL-1RII mRNA in the endometrial tissue was further evaluated by semiquantitative RT-PCR in 8 normal controls and 10 women with endometriosis (stages I and II). A representative RT-PCR and Southern blot analysis of IL-1RII mRNA in the endometrial tissue of women with endometriosis and of normal controls is shown in FIG. 23A. Levels of mRNA were significantly lower in the endometriosis group than in the control group (P<0.0062) (FIG. 23B), which corroborates the in situ hybridization data.

### Example 11

### Comparison of IL-1RII and IL-1 Concentration in Serum Between Healthy Women and Women Suffering from Endometriosis

Patients. Women were recruited into the study after they provided informed consent for a protocol approved by Saint-Francois d'Assise Hospital ethics committee on human research. Endometriosis was identified during laparoscopy for infertility and/or pelvic pain or for tubal ligation only. The stage of endometriosis was determined according to the revised classification of The American Fertility Society (American Fertility Society, 1985). Subjects with endometriosis (n=79) otherwise had no other pelvic pathology and were not taking any anti-inflammatory or hormonal medication at least 3 months before laparoscopy. Control subjects (n=38) were fertile women requesting tubal ligation and having no visible evidence of endometriosis at laparoscopy (Table 11). The cycle phase (proliferative or secretory) was determined according to the patients' cycle history and to the serum progesterone. Thirty three of endometriosis women and 17 of control subjects were in the proliferative phase of the menstrual cycle, whereas 46 of endometriosis women and 21 of control subjects were in the secretory phase.

TABLE 11
Serum source and clinical characteristics of subjects at laparoscopy

| Serum Source | Number of patients | Age (mean ± SD) |
|---|---|---|
| Controls | 38 | 34 ± 5 |
| Endometriosis | 79 | 32 ± 5 |
| Stage I | 36 | 32 ± 6 |
| Stage II | 27 | 31 ± 5 |
| Stages I and II | 63 | 32 ± 6 |
| Stages III and IV | 16 | 34 ± 4 |

Collection and Processing of blood Samples. Blood samples were drawn a few days before laparoscopy in sterile tubes containing ethylenediaminetetracetic acid and immediately centrifuged at 2000 g for 10 minutes at 4° C. The serum was then recovered, separated into small aliquots and stored at −80° C. until assay.

IL-1RII enzyme linked immunosorbent assay (ELISA). sIL-1RII concentrations in serum were measured using an ELISA developed in the laboratory. This assay uses respectively a mouse monoclonal anti-human IL-1RII antibody (R & D systems) for capture, a goat polyclonal anti-human IL-1RII antibody (R & D systems) for detection, peroxidase-conjugated rabbit anti-goat immunoglobulins (Zymed Laboratories, Inc. San Francisco, Calif.) and TMB (3,3′, 5,5′-tetramethylbenzidine) (Bio-Rad Laboratories Ltd, Mississauga, Ontario, Canada) as substrate for peroxidase. The optical density (OD) was determined at 450 nm, and sIL-1RII concentrations were calculated by interpolation from the standard curve.

IL-13 and IL-1α enzyme immunometric assay (EIA). The measurements were performed using EIA commercial kits, according to the manufacturer's instructions (Cayman Chemical, MI, USA).

Statistical analysis. Data are presented as mean±SEM. The unpaired t test was used to compare the levels of MCP-1 secretion or circulating cytokines in women with endometriosis and control subjects, and Bonferroni correction was applying for multiple comparisons. To compare the effect of one treatment on MCP-1 secretion, we used the paired t test. Differences were considered as statistically significant for P values<0.05.

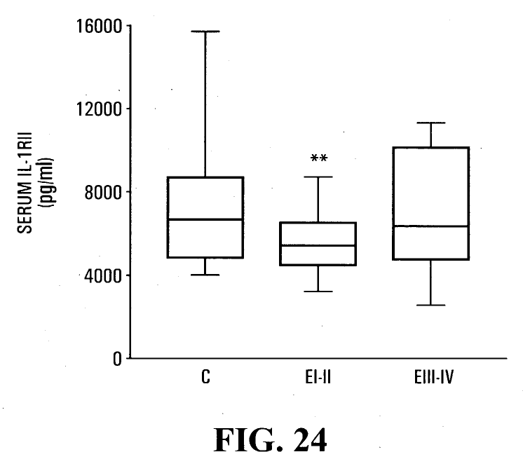

We measured sIL-1RII concentrations in the serum of normal controls and women with endometriosis stages I-II and III-IV. Statistical analysis of the results with the unpaired t test showed a significant decrease in the levels of sIL-1RII in the endometriosis group as compaired to the control group (P=0.0076). Furthermore, this decrease was found to be significant in the initial stages of endometriosis (stages I-II) (P=0.002) (FIG. 24). Statistical analysis of our data according to the menstrual cycle phase showed no significant difference in sIL-1RII levels between the proliferative phase and the secretory phase, neither in normal controls, nor in endometriosis stages I-II or III-IV. However, sIL-RII levels were significantly higher in endometriosis stages I-II as compared to normal controls both in the proliferative phase (P=0.0184) and the secretory phase (P=0.0261).

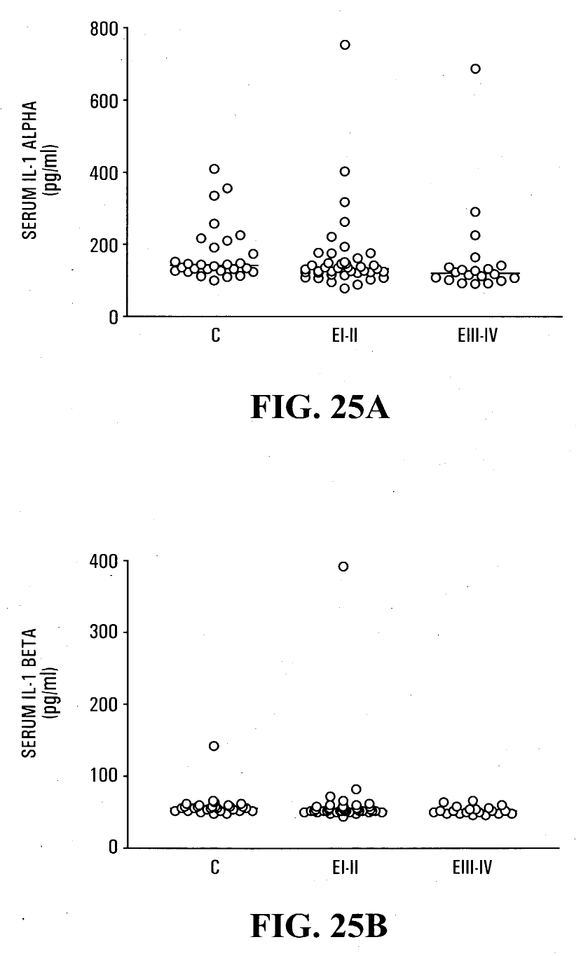

We then measured the circulating levels of IL-1 in its two forms IL-1α and IL-1β. FIG. 25 shows the distribution of IL-1α and IL-1β concentrations found in normal and endometriosis women according to the stage of the disease. Statistical analysis of data with the unpaired t test showed no significant difference in IL-1α or IL-1β concentrations between normal and endometriosis women, whether these latter were grouped together (P=0.955 and 0.667, respectively) or separated into 2 groups of endometriosis stages I-II (P=0.710 and 0.706, respectively) and III-IV (P=0.657 and 0.103, respectively).

### Example 12

### Comparison of MCP-1 Secretion by U937 Cells Between the Serum of Healthy Women and Women Suffering from Endometriosis

Patients. Women were recruited into the study after they provided informed consent for a protocol approved by Saint-Francois d'Assise Hospital ethics committee on human research. Endometriosis was identified during laparoscopy for infertility and/or pelvic pain or for tubal ligation only. The stage of endometriosis was determined according to the revised classification of The American Fertility Society (American Fertility Society, 1985). Subjects with endometriosis (n=79) otherwise had no other pelvic pathology and were not taking any anti-inflammatory or hormonal medication at least 3 months before laparoscopy. Control subjects (n=38) were fertile women requesting tubal ligation and having no visible evidence of endometriosis at laparoscopy (Table 11). The cycle phase (proliferative or secretory) was determined according to the patients' cycle history and to the serum progesterone. Thirty three of endometriosis women and 17 of control subjects were in the proliferative phase of the menstrual cycle, whereas 46 of endometriosis women and 21 of control subjects were in the secretory phase.

Collection and Processing of blood Samples. Blood samples were drawn a few days before laparoscopy in sterile tubes containing ethylenediaminetetracetic acid and immediately centrifuged at 2000 g for 10 minutes at 4° C. The serum was then recovered, separated into small aliquots and stored at −80° C. until assay.

MCP-1 enzyme-linked immunosorbent assay (ELISA). MCP-1 concentrations were measured using a previously described ELISA (Akoum A, et al, 1991). This assay uses a mouse monoclonal anti-human MCP-1 antibody (R & D systems) and a rabbit polyclonal anti-human MCP-1 antibody. This latter antibody does not cross-react with several cytokines that are closely related to MCP-1, including MCP-2, MCP-3, IL-8, the protein regulated on activation normal T expressed and secreted (RANTES) and the macrophage inflammatory protein-1α and β (MIP-1α and MIP-1β) (Hachicha M, et al, 1993).

Monocyte culture and biological assay. For these studies, we have used a hystiocytic cell line (U937). Cells were culture at 37° C., 5% CO₂ in RPMI medium supplement with 10% heat-inactivated fetal bovine serum (FBS) and 1% antibiotics, and incubated with 1 mM cyclic adenosine monophosphate (cAMP)(Sigma, St. Louis) for 48 hours to induce cell differentiation. Cells were harvested by centrifugation, then distributed in 24-well culture plates at 10⁶ cells/ml/well in FBS-free RPMI medium supplemented with different serum dilutions (5, 10 and 20%), and incubated in duplicate for 24 hours at 37° C. Culture supernatants were then collected and frozen at −80° C. until assay. The biological assay was performed on pooled sera from normal controls or from women with endometriosis according to endometriosis stage (I, II, and III-IV). An equal volume of serum was taken from all the patients included in each group. The effect of serum-induced MCP-1 secretion was also studied in the presence of human rIL-1RII (5 μg/ml) and human rIL-1ra (100 ng/ml) (R & D systems, Minneapolis, Minn.).

Statistical analysis. Data are presented as mean±SEM. The unpaired t test was used to compare the levels of MCP-1 secretion or circulating cytokines in women with endometriosis and control subjects, and Bonferroni correction was applying for multiple comparisons. To compare the effect of one treatment on MCP-1 secretion, we used the paired t test. Differences were considered as statistically significant for P values<0.05.

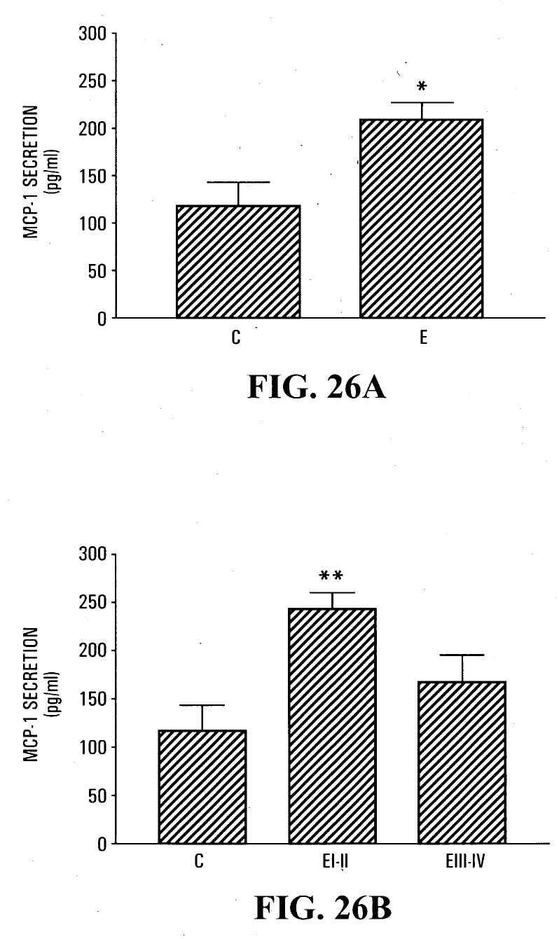

Considering the above results showing comparable levels of circulating IL-1 in normal and endometriosis women, but reduced levels of sIL-1RII in women having the disease, we further assessed the effect of peripheral blood serum from women with and without endometriosis on monocyte activation, by measuring MCP-1 secretion by U937 monocytic cells in response to these sera. As shown in FIG. 26, serum from women with endometriosis induced a higher secretion of MCP-1 by U937 cells than that from normal women (P=0.018). Furthermore, this effect appeared to be significant in the initial (P=0.002) rather than in the more advanced (P=0.238) stages of the disease.

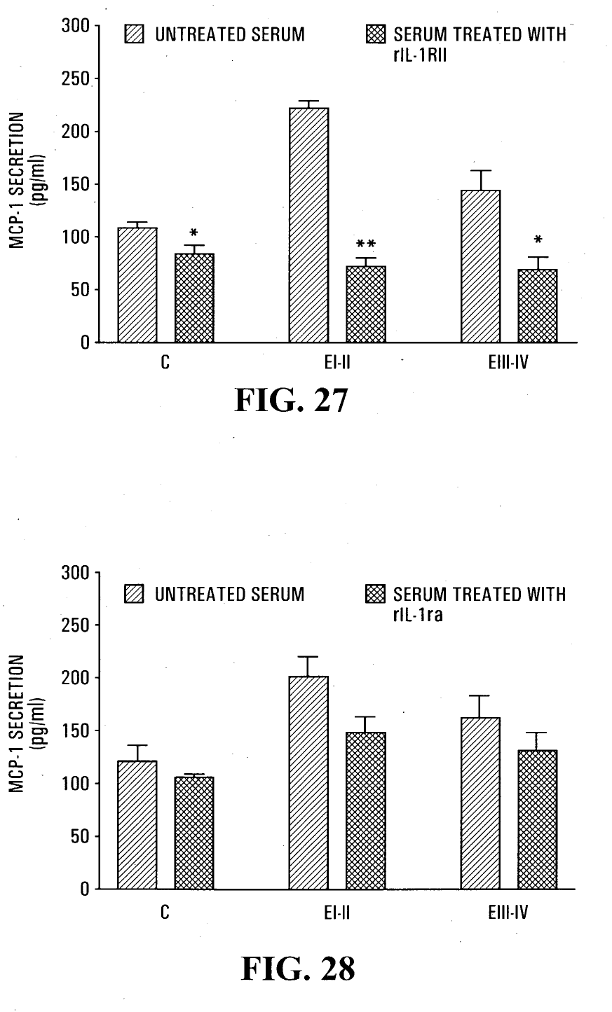

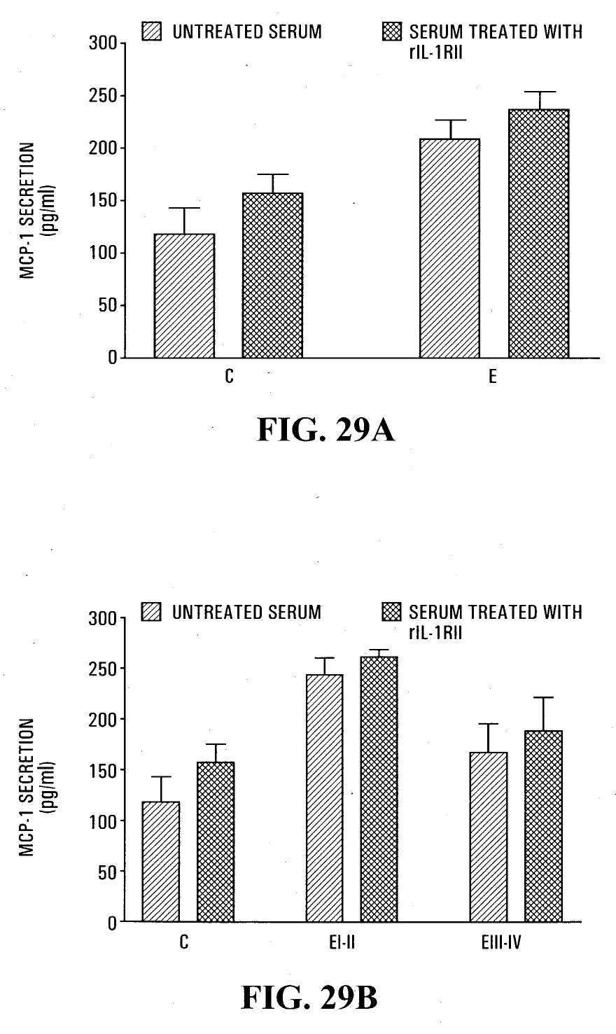

Human rIL-1RII (5 μg/ml) was then added to each pool of serum prior to incubation with U937 cells. As shown in FIG. 27, rIL-1RII significantly inhibited MCP-1 secretion by U937 cells in response to control and to endometriosis women-derived sera. However, the most significant inhibition of MCP-1 induced secretion was observed in endometriosis stage I and II(P=0.0008).

U937 cells were further incubated for 24 hours at 37° C. with each pool of serum to which recombinant human IL-1ra (100 ng/ml) was added. Our data depicted in FIG. 28 show a slight inhibition of MCP-1 secretion induced by normal as well as by endometriosis women-derived sera. It is noteworthy that IL-1ra inhibitory effect was, to some extent, more noticeable in serum from women with endometriosis stages I-II, but no statistically significant difference between IL-1ra-treated and untreated sera was found (P 0.101).

### Example 13

### Analysis of MIF Expression in Endometrial Tissue Collected from Healthy Women and from Women Suffering from Endometriosis

Endometrial tissues used in this study were obtained from women who provided informed consent for a research protocol approved by Saint-François d'Assise Hospital Ethics Committee on Human Research. Biopsies were immediately placed at 4° C. in sterile Hanks' Balanced Salt Solution (HBSS) (GIBCO BRL, Burlington, Ontario, Canada) containing 100 IU/mL penicillin, 100 μg/mL streptomycin, and 0.25 μg/mL amphotericin, transported to the laboratory where they were immediately washed in HBSS at 4° C. and snap-frozen on dry ice. Frozen tissues were directly homogenized with the use of a micro-scale tissue grinder (Kontes, Vineland, N.J., USA) in a buffer containing 0.5% Triton-X100, 10 mM HEPES (pH 7.4), 150 mM NaCl, 2 mM ethylene glycol tetra-acetic acid (EGTA), 2 mM ethylene diamine tetra-acetic acid (EDTA), 0.02% NaN3 (15) and a mixture of anti-proteases composed of 5 μM aprotinin, 63 μM leupeptin and 3 mM phenylmethylsulfonylfluoride (PMSF). Tissue homogenate was then incubated at 4° C. for 45 min under gentle shaking, and centrifuged at 11 000 g for 30 min to recover the soluble extract. Total protein concentration was determined using the Bio-Rad DC Protein Assay (Bio-Rad Laboratories Ltd., Mississauga, ON, Canada).

MIF concentration in tissue protein extracts was measured by ELISA. Briefly, this technique uses a capture mouse monoclonal anti-human-MIF antibody (210 ng/well) (R&D Systems), a rabbit polyclonal anti-human-MIF antibody for detection (1 μg/mL), alkaline phosphatase-conjugated goat anti-rabbit IgGs (1/5000 dilution) (Chemicon International Inc, Temecula, Calif., USA) and para-nitrophenyl phosphate as substrate (Sigma). The optical density (OD) was measured at 405 nm and MIF concentrations were extrapolated from a standard curve using recombinant human MIF (R&D Systems).

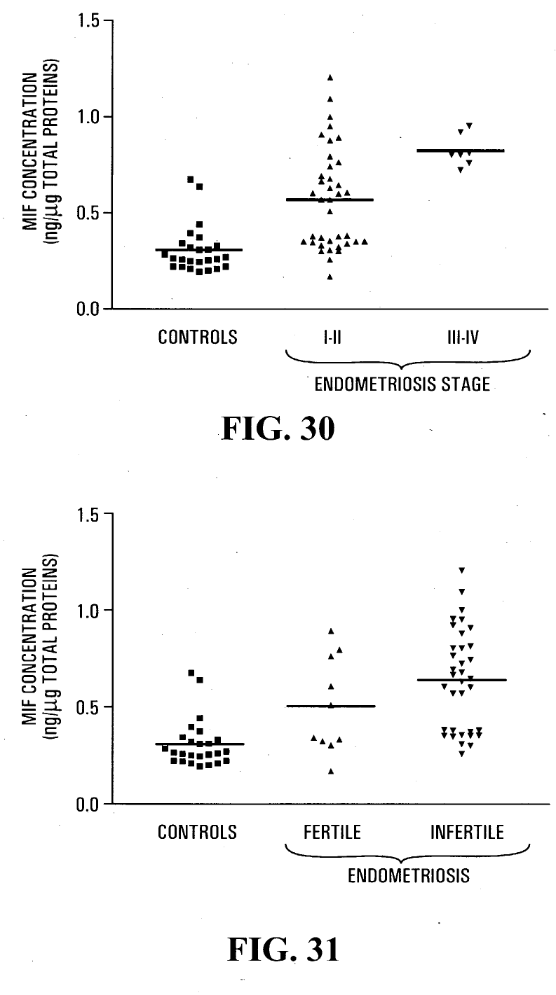

MIF concentrations were markedly higher in endometriosis patients as compared with normal controls (p<0.0001), and appeared to correlate with the severity of the disease (FIG. 30).

Both fertile and infertile women with endometriosis had significantly higher MIF concentrations than fertile women having a normal gynecological status. However, a more significant increase was found in infertile endometriosis patients (p<0.001), which in favor of MIF involvement in endometriosis-associated infertility (FIG. 31).

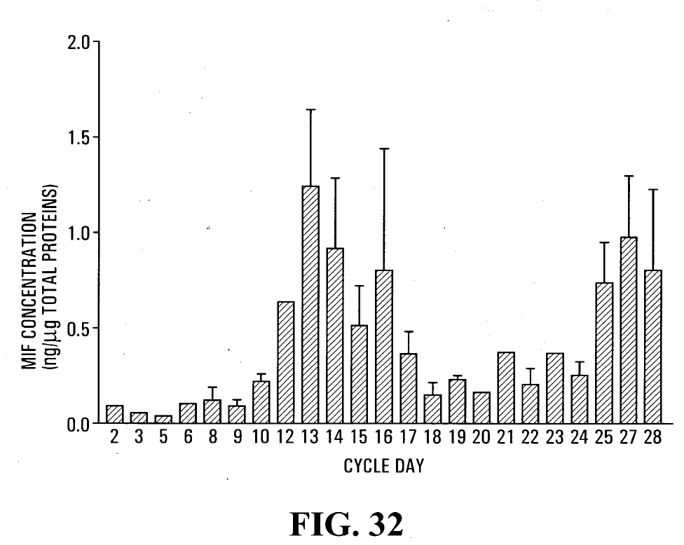

MIF protein expression in the endometrial tissue followed a regulated cycle phase-dependent pattern. Being elevated in the late-proliferative/early secretory phase of the menstrual cycle, MIF expression decreased in the mid-secretory phase before augmenting markedly again during the late secretory phase (FIG. 32).

### Example 14

### Human MIF and IL-1RII DNA and Polypeptide Sequences

Homo sapiens macrophage migration inhibitory factor (glycosylation-inhibiting factor) (MIF), mRNA gi|4505184|ref|NM_{—}002415.1|[4505184] (SEQ ID NO:7)

| | |
|---|---|
| 1 | accacagtgg tgtccgagaa gtcaggcacg tagctcagcg gcggccgcgg cgcgtgcgtc |
| 61 | tgtgcctctg cgcgggtctc ctggtccttc tgccatcatg ccgatgttca tcgtaaacac |
| 121 | caacgtgccc cgcgcctccg tgccggacgg gttcctctcc gagctcaccc agcagctggc |
| 181 | gcaggccacc ggcaagcccc cccagtacat cgcggtgcac gtggtcccgg accagctcat |
| 241 | ggccttcggc ggctccagcg agccgtgcgc gctctgcagc ctgcacagca tcggcaagat |
| 301 | cggcggcgcg cagaaccgct cctacagcaa gctgctgtgc ggcctgctgg ccgagcgcct |
| 361 | gcgcatcagc ccggacaggg tctacatcaa ctattacgac atgaacgcgg ccaatgtggg |
| 421 | ctggaacaac tccaccttcg cctaagagcc gcagggaccc acgctgtctg cgctggctcc |
| 481 | acccgggaac ccgccgcacg ctgtgttcta ggcccgccca ccccaacctt ctggtgggga |
| 541 | gaaataaacg gtttagagac t. |

Translation of coding sequence (98-445 above); human MIF protein (SEQ ID NO:8):

MPMFIVNTNVPRASVPDGFLSELTQQLAQATGKPPQYIAVHVVPDQLMAF
GGSSEPCALCSLHSIGKTGGAQNRSYSKLLCGLLAERLRISPDRVYINYY
DMNAANVGWNNSTFA

Homo sapiens interleukin 1 receptor, type II (IL1R2), transcript variant 1, mRNA gi|27894332|ref|NM_{—}004633.31 [27894332] (SEQ ID NO:9)

| | |
|---|---|
| 1 | cccgtgagga ggaaaaggtg tgtccgctgc cacccagtgt gagcgggtga caccacccgg |
| 61 | ttaggaaatc ccagctccca agagggtata aatccctgct ttactgctga gctcctgctg |
| 121 | gaggtgaaag tctggcctgg cagccttccc caggtgagca gcaacaaggc cacgtgctgc |
| 181 | tgggtctcag tcctccactt cccgtgtcct ctggaagttg tcaggagcaa tgttgcgctt |
| 241 | gtacgtgttg gtaatgggag tttctgcctt cacccttcag cctgcggcac acacaggggc |
| 301 | tgccagaagc tgccggtttc gtgggaggca ttacaagcgg gagttcaggc tggaagggga |
| 361 | gcctgtagcc ctgaggtgcc cccaggtgcc ctactggttg tgggcctctg tcagcccccg |
| 421 | catcaacctg acatggcata aaaatgactc tgctaggacg gtcccaggag aagaagagac |
| 481 | acggatgtgg gcccaggacg gtgctctgtg gcttctgcca gccttgcagg aggactctgg |
| 541 | cacctacgtc tgcactacta gaaatgcttc ttactgtgac aaaatgtcca ttgagctcag |
| 601 | agtttttgag aatacagatg ctttcctgcc gttcatctca tacccgcaaa ttttaacctt |
| 661 | gtcaacctct ggggtattag tatgccctga cctgagtgaa ttcacccgtg acaaaactga |
| 721 | cgtgaagatt caatggtaca aggattctct tcttttggat aaagacaatg agaaatttct |
| 781 | aagtgtgagg gggaccactc acttactcgt acacgatgtg gccctggaag atgctggcta |
| 841 | ttaccgctgt gtcctgacat ttgcccatga aggccagcaa tacaacatca ctaggagtat |
| 901 | tgagctacgc atcaagaaaa aaaaagaaga gaccattcct gtgatcattt cccccctcaa |
| 961 | gaccatatca gcttctctgg ggtcaagact gacaatcccg tgtaaggtgt ttctgggaac |
| 1021 | cggcacaccc ttaaccacca tgctgtggtg gacggccaat gacacccaca tagagagcgc |
| 1081 | ctacccggga ggccgcgtga ccgaggggcc acgccaggaa tattcagaaa ataatgagaa |
| 1141 | ctacattgaa gtgccattga tttttgatcc tgtcacaaga gaggatttgc acatggattt |
| 1201 | taaatgtgtt gtccataata ccctgagttt tcagacacta cgcaccacag tcaaggaagc |
| 1261 | ctcctccacg ttctcctggg gcattgtgct ggccccactt tcactggcct tcttggtttt |
| 1321 | ggggggaata tggatgcaca gacggtgcaa acacagaact ggaaaagcag atggtctgac |
| 1381 | tgtgctatgg cctcatcatc aagactttca atcctatccc aagtgaaata aatggaatga |
| 1441 | aataattcaa acacaaaaaa aaaaaaaaaa aaaaaaaaaa aaaa |

Translation of coding sequence (230-1426 above); human IL-1RII protein (SEQ ID NO:10):

MLRLYVLVMGVSAFTLQPAAHTGAARSCRFRGRHYKREERLEGEPVALRC
PQVPYWLWASVSPRTNLTWHKNDSARTVPGEEETRMWAQDGALWLLPALQ
EDSGTYVCTTRNASYCDKMSTELRVFENTDAFLPFISYPQILTLSTSGVL
VCPDLSEFTRDKTDVKIQWYKDSLLLDKDNEKFLSVRGTTHLLVHDVALE
DAGYYRCVLTFAHEGQQYNITRSIELRIKKKKEETIPVIISPLKTTSASL
GSRLTIPCKVFLGTGTPLTTMLWWTANDTHIESAYPGGRVTEGPRQEYSE
NNENYTEVPLIFDPVTREDLHMDFKCVVHNTLSFQTLRTTVKEASSTFSW
GIVLAPLSLAFLVLGGIWMHRRCKHRTGKADGLTVLWPHHQDFQSYPK

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

What is claimed is:

1. A method of assessing a reproduction-associated disease in a subject, said method comprising
(a) determining a test level of a parameter selected from the group consisting of:
(i) MIF protein;
(ii) MIF encoding RNA, and
(iii) MIF activity;
in a tissue or body fluid from said subject; and
(b) comparing said test level to an established standard; or to a corresponding level of said parameter in a tissue or body fluid of a control subject; or to a corresponding level of said parameter in a tissue or body fluid obtained from said subject at an earlier time; wherein an increase in said test level is indicative of reproduction-associated disease.

2. The method of claims 1, wherein the subject is a mammal.

3. The method of claim 1, wherein the suject is a human.

4. The method of claim 1, wherein the body fluid or tissue is endometrial tissue or a derivative thereof.

5. The method of claim 1, wherein the reproduction-associated disease is endometriosis or infertility.

6. A method of assessing endometrial receptivity in a subject, said method comprising determining, in said subject, a test level of a parameter selected from the group consisting of:
(i) MIF protein;
(ii) IL-1RII protein;
(iii) MIF encoding RNA;
(iv) IL-1RII encoding RNA;
(v) MIF activity; and
(vi) and IL-1RII activity;
wherein said test level is indicative of endometrial receptivity.

7. The method of claim 6, further comprising comparing said test level with an established standard, or to a corresponding level of said parameter from a control subject; or to a corresponding level of said parameter obtained from said subject at an earlier time, to obtain a comparison result.

8. The method of claim 7, further comprising predicting a window of implantation in accordance with said comparison result.

9. The method of claim 6, wherein the subject is in a secretory phase of a menstrual cycle.

10. The method of claim 6, wherein said test level is determined in a body fluid or a tissue obtained from said subject.

11. The method of claim 10, wherein the body fluid or the tissue is endometrial tissue or a derivative thereof.

12. A method of treating a reproductive-related disease in a subject, said method comprising:
(a) administering an effective amount of an IL-1RII or an IL-1RII-related compound to said subject;
(b) enhancing IL-1RII activity in said subject;
(c) enhancing IL-1RII expression in a cell or tissue of said subject; and
(d) any combination of (a) to (c).

13. The method of claim 12, wherein the subject is a mammal.

14. The method of claim 12, wherein the subject is a human.

15. The method of claim 12, wherein the cell is a endometrial cell, and wherein the tissue is an endometrial tissue.

16. A commercial package comprising means for assessing the level of a parameter selected from the group consisting of:
(i) MIF protein;
(ii) MIF encoding RNA; and
(iii) MIF activity;
in a tissue or body fluid; together with instructions for diagnosis, prognostication, or both, of reproduction-associated disease.

17. The commercial package of claim 16, further comprising a reference value or reference sample of said parameter.

18. The commercial package of claim 17, wherein the reference value is an established standard of said parameter; and wherein the reference sample is a corresponding level of said parameter in a tissue or body fluid of a control subject.

19. The commercial package of claim 16, wherein the body fluid or tissue is endometrial tissue or a derivative thereof.

20. A commercial package comprising means for assessing the level of a parameter selected from the group consisting of:
(i) MIF protein;
(ii) IL-1RII protein;
(iii) MIF encoding RNA;
(iv) IL-1RII encoding RNA;
(v) MIF activity; and
(vi) IL-1RII activity;
in a tissue or body fluid, together with instructions for the determination of the endometrial receptivity.

21. The commercial package of claim 20, further comprising a reference value or reference sample of said parameter.

22. The commercial package of claim 21, wherein the reference value is an established standard of said parameter; and wherein the reference sample is a corresponding level of said parameter in a tissue or body fluid of a control subject.

23. The commercial package of claim 20, wherein the body fluid or tissue is endometrial tissue or a derivative thereof.

24. A commercial package comprising an IL-1RII or an IL-1RII related compound together with instructions for treating a reproductive-related disease in a subject.

25. A method for lowering endometrial receptivity in a subject, said method comprising administering an effective amount of an IL-1RII or IL-1RII related compound to said subject.

26. A commercial package comprising an IL-1RII or an IL-1RII related compound together with instructions for lowering endometrial receptivity in a subject.


SEQUENCE LISTING (10 sequences)

SEQ ID NO: 1 - DNA, 21 bp, Artificial
  primer
tccatgtgcaaatcctctctt

SEQ ID NO: 2 - DNA, 21 bp, Artificial
  primer
tcctgccgttcatctcatacc

SEQ ID NO: 3 - DNA, 25 bp, Artificial
  primer
tgatgacatcaagaaggtggtgaag

SEQ ID NO: 4 - DNA, 23 bp, Artificial
  primer
tccttggaggccatgtgggccat

SEQ ID NO: 5 - DNA, 21 bp, Artificial
  primer
ctctccgagctcacccagcag

SEQ ID NO: 6 - DNA, 21 bp, Artificial
  primer
cgcgttcatgtcgtaatagtt

SEQ ID NO: 7 - DNA, 561 bp, Homo sapiens
  CDS (98)..(445)
accacagtggtgtccgagaagtcaggcacgtagctcagcggcggccgcggcgcgtgcgtctgtgcctctgcgcgggtctcctggtccttctgccatcatgccgatgttcatcgtaaacaccaacgtgccccgcgcctccgtgccggacgggttcctctccgagctcacccagcagctggcgcaggccaccggcaagcccccccagtacatcgcggtgcacgtggtcccggaccagctcatggccttcggcggctccagcgagccgtgcgcgctctgcagcctgcacagcatcggcaagatcggcggcgcgcagaaccgctcctacagcaagctgctgtgcggcctgctggccgagcgcctgcgcatcagcccggacagggtctacatcaactattacgacatgaacgcggccaatgtgggctggaacaactccaccttcgcctaagagccgcagggacccacgctgtctgcgctggctccacccgggaacccgccgcacgctgtgttctaggcccgcccaccccaaccttctggtggggagaaataaacggtttagagact

SEQ ID NO: 8 - protein, 115 aa, Homo sapiens
MPMFIVNTNVPRASVPDGFLSELTQQLAQATGKPPQYIAVHVVPDQLMAFGGSSEPCALCSLHSIGKIGGAQNRSYSKLLCGLLAERLRISPDRVYINYYDMNAANVGWNNSTFA

SEQ ID NO: 9 - DNA, 1484 bp, Homo sapiens
  CDS (230)..(1426)
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 10 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK